(19) United States
(12) Patent Application Publication
(10) Pub. No.: US 2023/0044649 A1
(43) Pub. Date: Feb. 9, 2023

(54) METHODS TO ENHANCE BIOAVAVAILABILITY OF ORGANIC SMALL MOLECULES AND DEPOSITED FILMS MADE THEREFROM

(71) Applicant: THE REGENTS OF THE UNIVERSITY OF MICHIGAN, Ann Arbor, MI (US)

(72) Inventors: Olga Shalev, Gilon (IL); Max Shtein, Ann Arbor, MI (US); Shreya Raghavan, Ann Arbor, MI (US); Geeta Mehta, Ann Arbor, MI (US)

(21) Appl. No.: 17/840,276

(22) Filed: Jun. 14, 2022

Related U.S. Application Data
(63) Continuation of application No. 15/579,871, filed on Dec. 5, 2017, now abandoned.
(60) Provisional application No. 62/171,702, filed on Jun. 5, 2015.

(51) Int. Cl. A61K 9/70; A61K 31/192; A61K 31/522; C23C 14/12; A61K 31/138; A61K 31/167; A61L 31/16; A61K 45/06; A61K 31/277; A61K 31/352; A61L 27/54; C23C 14/22; C23C 14/04; A61K 31/275; A61K 31/65

(52) U.S. Cl. CPC A61K 9/7007; A61K 31/192; A61K 31/522; C23C 14/12; A61K 31/138; A61K 31/167; A61L 31/16; A61K 45/06; A61K 31/277; A61K 31/352; A61L 27/54; C23C 14/228; C23C 14/04; A61K 31/275; A61K 31/65; B05D 1/60

(57) ABSTRACT

Solid films and articles having a surface with discrete regions patterned with a deposited low molecular weight organic compound, such as pharmaceutical actives and new chemical entities, are provided. The organic compound may be present at ≥ about 99 mass % in the one or more discrete regions and may be crystalline or amorphous. The deposited organic compound may be deposited as a film having high surface area. The deposited organic compound exhibits enhanced solubility and bioavailability, by way of non-limiting example. Methods of organic vapor jet printing deposition method of such a low molecular weight organic compound in an inert gas stream are also provided.

### CROSS-REFERENCE TO RELATED APPLICATIONS

This application claims the benefit of U.S. Provisional Application No. 62/171,702, filed on Jun. 5, 2015. The entire disclosure of the above application is incorporated herein by reference.

### FIELD

The present disclosure relates to a pure deposited film of low molecular weight organic compounds (e.g., a pharmaceutical active ingredient or new chemical entity), where such deposited low molecular weight organic compounds have enhanced bioavailability and solubility. Methods and apparatuses of depositing a low molecular weight organic compound via deposition process, such as organic vapor jet printing deposition methods and apparatuses, are also provided.

### BACKGROUND

This section provides background information related to the present disclosure which is not necessarily prior art.

Solutions of small molecular organic compounds are used extensively in many industries: food, cosmetics/perfume, pharmaceutical, organic electronics, printing and paints, by way of non-limiting example. Aqueous solubility is an especially important factor in controlling bioavailability of active pharmaceutical ingredients (APIs). Thus, the pharmaceutical industry faces many challenges. For example, more than 40% of newly discovered drugs/new chemical entities (NCE) suffer from low solubility and dissolution rates, making them less favorable candidates for further research. This problem is especially important for substances with low solubility and high permeability (class II type according to the Biopharmaceutics Classification System). Existing methods for solubility enhancement usually include physical modifications: particle size reduction, modification of crystal habit, drug suspensions, solid dispersions, solid solutions and cryogenic techniques; chemical modifications: change of pH, use of buffer, salt formation, complexation and other methods like use of surfactants, cosolvency, hydrotrophy and novel excipients.

Particle size reduction approaches leverage the fact that solubility of the drug intrinsically depends on drug particle size: as particle size decreases, surface area to volume ratio increases, enhancing interaction with the solvent and resulting in improved solvation. Common methods for particle size reduction, such as spray drying, comminution, micronization and nanonization introduce physical stress upon the drug particles and have the potential to degrade sensitive NCE molecules and/or cause particle aggregation. In addition, these techniques usually require more complex processing techniques, including additional processing stages, like sifting and dividing, into specific dosages. Further, particle size reduction is not necessarily feasible for high potency drugs, where sub-microgram dosages are needed, or for newly developed drugs and drug candidates where large amounts (kilograms) are not yet available.

For example, nanonization is a well-known approach to enhance API powder bioavailability. As noted above, because dissolution process is dictated by surface area to volume ratio of a solute, decreasing particle size results in larger surface area and higher dissolution rate. However, nanonization has a number of disadvantages. First, mechanical methods such as powder milling and high pressure homogenization (HPH) are energy- and time-consuming. Second, the resulting nanoparticles may lack storage stability and controlled release. Third, formulating with nanoparticles is challenging since homogeneity and stability are difficult to achieve due to particles agglomeration and changes in crystallinity.

During initial discovery stages, these NCE compounds are often added to cell culture in organic solvent (e.g., dimethyl sulfoxide—DMSO) solutions. Initial drug testing involves dissolution of drug in organic solvents, e.g., DMSO, which might provide inaccurate estimation of drug efficacy and bioavailability. More specifically, solvents like DMSO exaggerate solubility of drug molecules, affect cell membrane permeability, and potentially lead to the selection of “undruggable” NCEs. Additionally, the lack of rapid phase screening methods combined with limited drug amounts often leads to a powder being used “as is,” leading to higher attrition rates in drug discovery. Even after efficacy is established in vitro, later stages of drug development involve chemical or physical modifications to improve solubility limits and dissolution kinetics.

Thus, in conventional processes, to achieve a given concentration of organic solute in original powder form, the required amount of powder is immersed directly in the solvent and dissolved until all powder particles are separated into solvated molecules. This process is especially challenging for low solubility substances, where dissolution rates are very slow. Thus, powder particle sizes may be reduced (via milling or other methods) and the solution is usually heated to enhance dissolution rates. This approach can be both time and energy consuming, as well as potentially damaging to compounds/solvent.

An additional drawback of direct immersion of powder solute in the solvent is when the actual required concentration of a compound or solution volume is very low. For instance, if required concentration is on the order of micromoles, and a required volume is 10 ml, the required weight of 200 g/mole material would be on the order of micrograms. This weight is not feasible to measure accurately for a precursor powder; therefore a higher concentration of solution is made with subsequent dilution with additional amount of solvent. This process is undesirable from both economical and safety standpoint (when dealing with organic solvent).

A new streamlined approach for enhancing solubility and bioavailability, as well as improved ability to screen compounds for solubility and efficacy without the use of organic solvents, would be highly desirable and substantially accelerate drug development cycles and improve pharmaceutical compositions.

### SUMMARY

This section provides a general summary of the disclosure, and is not a comprehensive disclosure of its full scope or all of its features.

In certain variations, the present disclosure provides a solid film comprising greater than or equal to about 99 mass % of a deposited low molecular weight organic active ingredient compound having a molecular weight of less than or equal to about 1,000 g/mol. The low molecular weight organic active ingredient compound may be a pharmaceutical active or a new chemical entity. The deposited low molecular weight organic compound has an enhanced solubility as compared to a powder non-deposited form of the low molecular weight organic compound.

In other variations, the present disclosure provides an article comprising a surface of a solid substrate having one or more discrete regions patterned with a deposited low molecular weight organic compound having a molecular weight of less than or equal to about 1,000 g/mol. In certain aspects, the deposited low molecular weight organic compound is present at greater than or equal to about 99 mass % in the one or more discrete regions.

In yet other variations, the present disclosure provides an article comprising a pharmaceutically acceptable substrate defining a surface. The article also comprises a deposited solid low molecular weight pharmaceutical active ingredient having a molecular weight of less than or equal to about 1,000 g/mol. The deposited solid low molecular weight pharmaceutical active ingredient is present at greater than or equal to about 99 mass % in one or more discrete regions on the surface of the pharmaceutically acceptable substrate.

In certain other variations, the present disclosure provides an article comprising a solid deposited film comprising a pharmaceutical composition. The pharmaceutical composition comprises at least one low molecular weight organic compound having a molecular weight of less than or equal to about 1,000 g/mol.

In other variations, the present disclosure provides a solvent-free vapor deposition method that comprises depositing a low molecular weight organic compound, for example, having a molecular weight of less than or equal to about 1,000 g/mol, on one or more discrete regions of a substrate in a process that is substantially free of solvents. The process may be selected from the group consisting of: vacuum thermal evaporation (VTE), organic vapor jet printing (OVJP), organic vapor phase deposition (OVPD), organic molecular beam deposition (OMBD), molecular jet printing (MoJet), organic vapor jet printing (OVJP), and organic vapor phase deposition (OVPD). A deposited low molecular weight organic compound is present at greater than or equal to about 99 mass % in the one or more discrete regions.

In yet other variations, the present disclosure provides an organic vapor jet printing deposition method comprising entraining a low molecular weight organic compound in an inert gas stream by heating a source of a solid low molecular weight organic compound to sublimate the low molecular weight organic compound. The inert gas stream is passed over, by, or through the source. The low molecular weight organic compound is directed through a nozzle towards a cooled target. Then, the low molecular weight organic compound is condensed as it contacts the cooled target.

In certain other variations, the present disclosure provides a method for rapid dissolution of low molecular weight organic compounds. The method comprises passing a gas stream comprising an inert gas past a heated source of the low molecular weight organic compound. The low molecular weight organic compound is volatilized and entrained in the gas stream. Then, the low molecular weight organic compound is deposited into a liquid comprising one or more solvents by passing the gas stream through a nozzle towards the liquid. In this manner, the deposited low molecular weight organic compound is dissolved in the liquid.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIGS. 1(a)-1(d) show schematics of organic vapor jet printing deposition techniques and apparatuses according to certain aspects of the present disclosure. FIG. 1(a) shows an organic vapor jet deposition (OVJP) system for small molecular drugs deposition system. FIG. 1(b) shows a mixed layer OVJP deposition mode—the system comprises multiple sources of material to be evaporated that are later mixed in the main jet stream. FIG. 1(c) shows a multilayer mode of OVJP deposition for forming distinct materials in one or more discrete regions on a surface of a substrate, where the distinct materials may overlap with one another. FIG. 1(d) shows a select patterning mode for OVJP deposition to deposit distinct materials.

FIGS. 2(a)-2(b) show a schematic of a specialized design for a source or organic material used in an OVJP deposition technique according to certain aspects of the present disclosure. FIG. 2(a) shows the evaporation source comprises an outer casing (“boat case”) and a ceramic foam plug that enables the evaporated molecules to be sublimed and carried through the porous foam in a highly reproducible manner. FIG. 2(b) shows an example of evaporation source implementation. The boat case is made of quartz and the ceramic foam is silicon carbide with porosity of 80 pores per inch (ppi) from Ultramet. The powder to be evaporated is placed between porous foam disks, or between a foam disk and a portion of quartz wool. The source can be reused after washing out the organic powder with appropriate solvents.

FIG. 3 shows a variety of examples of printed pharmaceutical organic compounds deposited via organic vapor jet printing deposition techniques in accordance with certain aspects of the present disclosure.

FIGS. 4(a)-4(b) show an example of a printed pharmaceutical film having a deposited organic compound (BAY 11-7082) in comparative testing in a deposited film in accordance with certain aspects of the present disclosure as compared to a comparative DMSO preparation for assessing biological efficacy.

FIGS. 5(a)-5(c) show schematics of organic vapor jet printing deposition techniques and an apparatus according to certain alternative aspects of the present disclosure. FIG. 5(a) shows a schematic of rapid dissolution system for low molecular weight organic compounds according to certain aspects of the present disclosure. FIGS. 5(b)-5(c) show an example of fluorescein molecule jetted into phosphate buffer saline solution of 2 ml. Jetting conditions: Carrier gas: nitrogen. Carrier gas flow rate: 200 sccm. Source temperature: 300° C., substrate temperature: 20° C., nozzle tip inner diameter: 0.5 mm, nozzle tip-liquid surface separation distance: 20 mm. In FIG. 5(c), the concentration varies with jetting duration. Concentration is measured by fluorescence spectroscopy calibrated with dissolved fluorescein powder.

FIGS. 6(a)-6(r) shows surface morphology of solid printed films for caffeine, tamoxifen, BAY 11-7082, paracetamol, ibuprofen, and fluorescein. FIGS. 6(a)-6(f) show chemical structures of the tested compounds. FIGS. 6(g)-6(l) show deposited film morphologies after jetting in accordance with the certain aspects of the present teachings. FIGS. 6(m)-6(r) show original microstructure of powders of the compounds.

FIGS. 7(a)-7(h) shows drug films prepared in accordance with certain aspects of the present disclosure as compared to powders of the same drugs, along with structural characterizations. FIG. 7(a) shows ultra performance liquid chromatography results (UPLC) for caffeine powder and caffeine deposited film according to certain aspects of the present teachings. FIG. 7(b) shows UPLC for tamoxifen powder and deposited film. FIG. 7(c) shows UPLC of BAY 11-7082 powder and deposited film. FIG. 7(d) shows UPLC of paracetamol powder and deposited film. FIG. 7(e) shows X-Ray Diffraction (XRD) of caffeine powder and deposited film, with corresponding average crystal size. FIG. 7(f) shows XRD of tamoxifen powder and deposited film, with corresponding average crystal size. FIG. 7(g) shows XRD of BAY 11-7082 powder and deposited film. FIG. 7(h) shows XRD of paracetamol powder and deposited film, with corresponding average crystal size.

FIGS. 8(a)-8(d) demonstrate examples of different coating modes of fluorescein on different substrates in accordance with certain aspects of the present disclosure. FIG. 8(a) shows a solid deposited film of fluorescein on an acrylic polymer wound care patch, FIG. 8(b) FIG. 8(a) shows a solid deposited film of fluorescein on a pullulan-based film, FIG. 8(a) shows a solid deposited film of fluorescein on stainless steel microneedles, and FIG. 8(a) shows a solid deposited film of fluorescein on a borosilicate glass slide.

FIGS. 9(a)-9(b) show controlled release of printed fluorescein films prepared in accordance with certain aspects of the present disclosure. FIG. 9(a) shows a dissolution profile of printed fluorescein films of varying thickness and constant area. An inset in FIG. 9(a) shows dependence of (1−exp(−kt)) on film thickness. FIG. 9(b) shows a dissolution profile of printed fluorescein films with varying diameter and constant thickness. The dotted lines are experimental. Solid lines are predicted theoretical values. Inset of FIG. 9(b) shows films dissolution rate versus film area.

FIGS. 10(a)-10(c) show comparative dissolution profiles of films and powders. FIG. 10(a) shows a dissolution profile of fluorescein film and an original powder in deionized water. The dotted lines are experimental values. The solid lines are theoretical prediction for films and powders. FIG. 10(b) shows dissolution profiles of ibuprofen film and original powder in an aqueous HCl buffer pH 1.2 solution. The dotted line shows experimental values. The solid lines show theoretical prediction for film and powder. FIG. 10(c) shows dissolution profiles of tamoxifen film and original powder in acetate buffer pH 4.9 solution. The dotted line shows experimental values. The solid lines show theoretical prediction for film and powder.

FIG. 11 shows a schematic of drug application for a cancer cell growth study.

FIGS. 12(a)-12(d) demonstrate enhancement in biological efficacy of deposited films prepared in accordance with certain aspects of the present disclosure as compared to a conventional formulation. FIG. 12(a) shows an MCF7 cell treatment curve with tamoxifen (solid line—eye guide). FIG. 12(b) shows an OVCAR3 cell treatment curve with tamoxifen (solid line—eye guide). FIG. 12(c) shows an MCF7 cell treatment curve with BAY 11-7082 (solid line—eye guide). FIG. 12(d) shows OVCAR3 cell treatment curve with BAY 11-7082 (solid line—eye guide).

FIG. 13 shows a chart of specific film surface area as a function of deposited film area for different printed films weights.

Corresponding reference numerals indicate corresponding parts throughout the several views of the drawings.

### DETAILED DESCRIPTION

Example embodiments are provided so that this disclosure will be thorough, and will fully convey the scope to those who are skilled in the art. Numerous specific details are set forth such as examples of specific compositions, components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, elements, compositions, steps, integers, operations, and/or components, but do not preclude the presence or addition of one or more other features, integers, steps, operations, elements, components, and/or groups thereof. Although the open-ended term “comprising,” is to be understood as a non-restrictive term used to describe and claim various embodiments set forth herein, in certain aspects, the term may alternatively be understood to instead be a more limiting and restrictive term, such as “consisting of” or “consisting essentially of.” Thus, for any given embodiment reciting compositions, materials, components, elements, features, integers, operations, and/or process steps, the present disclosure also specifically includes embodiments consisting of, or consisting essentially of, such recited compositions, materials, components, elements, features, integers, operations, and/or process steps. In the case of “consisting of,” the alternative embodiment excludes any additional compositions, materials, components, elements, features, integers, operations, and/or process steps, while in the case of “consisting essentially of,” any additional compositions, materials, components, elements, features, integers, operations, and/or process steps that materially affect the basic and novel characteristics are excluded from such an embodiment, but any compositions, materials, components, elements, features, integers, operations, and/or process steps that do not materially affect the basic and novel characteristics can be included in the embodiment.

Any method steps, processes, and operations described herein are not to be construed as necessarily requiring their performance in the particular order discussed or illustrated, unless specifically identified as an order of performance. It is also to be understood that additional or alternative steps may be employed, unless otherwise indicated.

When a component, element, or layer is referred to as being “on,” “engaged to,” “connected to,” or “coupled to” another element or layer, it may be directly on, engaged, connected or coupled to the other component, element, or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various steps, elements, components, regions, layers and/or sections, these steps, elements, components, regions, layers and/or sections should not be limited by these terms, unless otherwise indicated. These terms may be only used to distinguish one step, element, component, region, layer or section from another step, element, component, region, layer or section. Terms such as “first,” “second,” and other numerical terms when used herein do not imply a sequence or order unless clearly indicated by the context. Thus, a first step, element, component, region, layer or section discussed below could be termed a second step, element, component, region, layer or section without departing from the teachings of the example embodiments.

Spatially or temporally relative terms, such as “before,” “after,” “inner,” “outer,” “beneath,” “below,” “lower,” “above,” “upper,” and the like, may be used herein for ease of description to describe one element or feature's relationship to another element(s) or feature(s) as illustrated in the figures. Spatially or temporally relative terms may be intended to encompass different orientations of the device or system in use or operation in addition to the orientation depicted in the figures.

Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters.

In addition, disclosure of ranges includes disclosure of all values and further divided ranges within the entire range, including endpoints and sub-ranges given for the ranges.

Example embodiments will now be described more fully with reference to the accompanying drawings.

The present disclosure provides a new streamlined approach for enhancing solubility and bioavailability of organic compounds, especially those that are new chemical entities (NCE) for drug discover or pharmaceutical compounds. In various aspects, the compositions, articles, and methods of the present teachings provide an improved ability to screen compounds for solubility and efficacy without the use of organic solvents, which can substantially accelerate drug development cycles and improve pharmaceutical compositions.

In certain aspects, the present disclosure provides materials and processes for continuous manufacturing and personalized dosing approaches of active ingredients. In various aspects, the present disclosure provides a solid film comprising a low molecular weight organic compound. In certain aspects, a low molecular weight compound may have a molecular weight of less than or equal to about 1,000 g/mol, optionally less than or equal to about 900 g/mol, optionally less than or equal to about 800 g/mol, optionally less than or equal to about 700 g/mol, optionally less than or equal to about 600 g/mol, optionally less than or equal to about 500 g/mol, optionally less than or equal to about 400 g/mol, optionally less than or equal to about 300 g/mol, and in certain variations, optionally less than or equal to about 200 g/mol. In certain variations, the low molecular weight compound may have a molecular weight of greater than or equal to about 100 g/mol to less than or equal to about 900 g/mol. The solid film may comprise a plurality of low molecular weight organic compounds. In certain variations, the low molecular weight organic compound is an active compound, such as a pharmaceutical active compound or a new chemical entity (a compound being investigated for potential pharmacological or bioactivity), as will be described further below. However, in alternative variations, the low molecular weight organic compound may be a nutritional or food compound, a nutraceutical compound, a cosmetic or personal care compound, a fragrance compound, a colorant or dye, an ink, a paint, and the like, by way of non-limiting example.

The present disclosure thus provides a solid film, for example, a deposited low molecular weight organic compound, such as a pharmaceutical active agent or a new chemical entity, patterned on a surface of a substrate. In certain variations, the surface has a continuous surface coating or film of the organic compound, while in other variations, the organic compound may be applied to select discrete regions of the surface. High quality films or coatings of low molecular organic compounds are formed by the processes according to certain aspects of the present disclosure that have high purity levels. For example, in certain variations, a purity level in one or more regions where of the low molecular weight compound is deposited may be greater than or equal to about 90% by mass of the low molecular weight compound, optionally greater than or equal to about 95% by mass, optionally greater than or equal to about 97% by mass, optionally greater than or equal to about 98% by mass, and in preferred aspects, optionally greater than or equal to about 99% by mass, optionally greater than or equal to about 99.5% by mass, optionally greater than or equal to about 99.7% by mass, and in certain variations, greater than or equal to about 99.99% by mass purity concentration. In certain variations, multiple low molecular weight compounds are present that together or cumulatively have the same purity levels. The deposited solid film may have a surface feature morphology ranging from molecularly flat to high surface area (e.g., a nanostructured surface) with feature sizes in the micrometer or nanometer regimes. Such a surface patterned with an organic compound enhances the solubility of medicinal organic compounds and substances, both at initial research stages and at the production level.

In certain aspects, methods of achieving solid films with high levels of purity and solubility are provided. For example, in certain variations, a solvent-free vapor deposition method is provided that includes depositing a low molecular weight organic compound on one or more discrete regions of a substrate in a process that is substantially free of solvents. By “substantially free” it is meant that solvent compounds or species are absent to the extent that undesirable and/or detrimental effects are negligible or nonexistent. In certain aspects, a vapor deposition process that is substantially free of solvents has less than or equal to about 0.5% by weight, optionally less than or equal to about 0.1% by weight, and in certain preferred aspects, 0% by weight of the undesired solvent species present during the deposition process.

A deposited low molecular weight organic compound may then be present at high purity levels, for example, at greater than or equal to about 99 mass % as described above, in the one or more discrete regions. The process for depositing the low molecular weight organic compound may be selected from the group consisting of: vacuum thermal evaporation (VTE), organic vapor jet printing (OVJP), organic vapor phase deposition (OVPD), organic molecular beam deposition (OMBD), molecular jet printing (MoJet), organic vapor jet printing (OVJP), and organic vapor phase deposition (OVPD).

In certain aspects, such a method may include entraining the low molecular weight organic compound in an inert gas stream or vacuum that is substantially free of any solvents prior to the depositing. An inert gas stream can comprise one or more generally nonreactive compounds, such as nitrogen, argon, helium, and the like. In certain variations, the inert gas stream comprises nitrogen.

Because many low molecular weight organic compounds, such as small molecular medicines, have sufficiently high vapor pressures (e.g., from about 1 Pa to about 100 Pa) and relatively low evaporation enthalpies (e.g., 100-300 kJ/mole), high evaporation rates (on the order of grams/(sec*m²)) can be achieved at temperatures of 100°−500° C., without reaching the temperature range where degradation of the compound can occur, even when evaporating at atmospheric pressure. Any process/system that enables deposition of molecular material onto a substrate from a vapor phase, where a source of the molecular material is a solid that evaporates or sublimates, can be used for forming the deposited low molecular weight organic compound pharmaceutical substances. This includes, but is not limited to: vacuum thermal evaporation (VTE), organic vapor phase deposition (OVPD), organic molecular beam deposition (OMBD), and molecular jet printing (MoJet).

However, the processes are not limited to solid sources of the low molecular weight compound. In certain aspects, prior to the entraining, the low molecular weight organic compound is in a form selected from the group consisting of: a powder, a pressed pellet, a porous material, and a liquid. In certain aspects, prior to the entraining, the low molecular weight organic compound is dispersed in pores of a porous material. In other aspects, prior to the entraining, the low molecular weight organic compound is dispersed in a liquid bubbler through which the inert gas stream passes. In yet other aspects, the entraining of the low molecular weight organic compound in the inert gas stream or vacuum is conducted by heating a source of a solid low molecular weight organic compound to sublimate or evaporate the low molecular weight organic compound. The methods of deposition result in the low molecular weight organic compound being deposited onto the one or more discrete regions at a loading density of greater than or equal to about 1×10⁻⁴ g/cm² to less than or equal to about 1 g/cm², in certain variations.

A parameter of the deposition process may be adjusted to control or affect a morphology, a degree of crystallinity, or both the morphology and the degree of crystallinity of the deposited solid low molecular weight organic compound. The parameter is selected from the group consisting of: system pressure, a flow rate of the inert gas stream, a composition of the inert gas, a temperature of a source of the low molecular weight organic compound, a composition of the substrate, a surface texture of the substrate, a temperature of the substrate, and combinations thereof.

In certain aspects, a specific surface area of the deposited low molecular weight organic compound is greater than or equal to about 0.001 m²/g to less than or equal to about 1,000 m²/g. The deposited low molecular weight organic compound may be amorphous. When the deposited low molecular weight organic compound is amorphous, it may further define interconnected particles having an average particle size (e.g., average particle diameter) of greater than or equal to about 2 nm to less than or equal to about 200 nm. In other aspects, the deposited low molecular weight organic compound is crystalline or polycrystalline. In such variations, an average crystal size or domain may be greater than or equal to about 2 nm to less than or equal to about 200 nm.

In certain aspects, the one or more discrete regions on which the low molecular weight organic compound is deposited are continuous so that a solid film is formed on the surface of the substrate. In certain variations, the one or more discrete regions of the surface have a high surface area morphology, which may optionally define one or more nanostructures or microstructures. In certain variations, an average thickness of the deposited low molecular weight organic compound in the one or more discrete regions of a surface of a substrate may be less than or equal to about 300 nm and an average surface roughness (Rₐ) may be less than or equal to about 100 nm. Thus, for solid deposited films with a thickness of 200±100 nm, the films are flat (roughness <100 nm). Starting with a thickness of around 200±100 nm, undulations occur in a solid deposited film, which further produce and define nanostructures. “Nano-sized” or “nanometer-sized” as used herein are generally understood by those of skill in the art to have at least one spatial dimension that is less than about 50 μm (i.e., 50,000 nm) and optionally less than about 10 μm (i.e., 10,000 nm). In certain aspects, an average thickness of the deposited low molecular weight organic compound in the one or more discrete regions is greater than or equal to about 300 nm and the deposited low molecular weight organic compound defines a nanostructured surface having a plurality of nanostructures having a major dimension of greater than or equal to about 5 nm to less than or equal to about 10 μm. The resulting morphology depends on thermophysical properties of the low molecular weight organic compound, the substrate material and deposition conditions. The plurality of nanostructures may have a shape selected from the group consisting of: needles, tubes or cylinders, rods, platelets, round particles (although they need not be perfectly round or circular), droplets, fronds, tree-like or fern-like structures, fractals, hemispheres, puddles, interconnected puddles, islands, interconnected islands, and combinations thereof. The shape of nanostructures formed depends on the low molecular weight organic compound being deposited, as well as the deposition process conditions, and film thickness.

In certain variations, a purity level of the deposited low molecular weight organic compound in the one or more discrete regions is any of those described previously, for example, greater than or equal to about 99.5 mass %. Suitable low molecular weight organic compounds, which may be pharmaceutical active ingredients or new chemical entities, may include by way of non-limiting example, various drugs or potential drugs (e.g., new chemical entities), including anti-proliferative agents; anti-rejection drugs; anti-thrombotic agents; anti-coagulants; antioxidants; free radical scavengers; nucleic acids; saccharides; sugars; nutrients; hormones; cytotoxin; hormonal agonists; hormonal antagonists; inhibitors of hormone biosynthesis and processing; antigestagens; antiandrogens; anti-inflammatory agents; non-steroidal anti-inflammatory agents (NSAIDs); antimicrobial agents; antiviral agents; antifungal agents; antibiotics; chemotherapy agents; antineoplastic/anti-miotic agents; anesthetic, analgesic or pain-killing agents; antipyretic agents, prostaglandin inhibitors; platelet inhibitors; DNA de-methylating agents; cholesterol-lowering agents; vasodilating agents; endogenous vasoactive interference agents; angiogenic substances; cardiac failure active ingredients; targeting toxin agents; and combinations thereof. The description of these suitable organic compounds/pharmaceutical active ingredients/new chemical entities is merely exemplary and should not be considered as limiting as to the scope of compounds or active ingredients which can be applied to a surface according to the present disclosure, as all suitable organic molecules and/or active ingredients known to those of skill in the art for these various types of compositions are contemplated. Furthermore, an organic compound may have various functionalities and thus, can be listed in an exemplary class above; however, may be categorized in several different classes of active ingredients.

Various suitable active ingredients are disclosed in Merck Index, An Encyclopedia of Chemicals, Drugs, and Biologicals, Thirteenth Edition (2001) by Merck Research Laboratories and the International Cosmetic Ingredient Dictionary and Handbook, Tenth Ed., 2004 by Cosmetic Toiletry and Fragrance Association, and at http://www.drugbank.ca/, the relevant portions of each of which are incorporated herein by reference. Each additional reference cited or described herein is hereby expressly incorporated by reference in its respective entirety. In certain variations, the low molecular weight organic compound is an active ingredient compound selected from the group: caffeine, (E)-3-(4-Methylphenylsulfonyl)-2-propenenitrile, fluorescein, paracetamol, ibuprofen, tamoxifen, and combinations thereof. BAY 11-7082 ((E)-3-(4-Methylphenylsulfonyl)-2-propenenitrile) selectively and irreversibly inhibits transcription factor NF-κB activation (which otherwise regulates expression of inflammatory cytokines, chemokines, immunoreceptors, and cell adhesion molecules) and can inhibit TNF-α-induced surface expression of adhesion molecules ICAM-1, VCAM-1, and E-selectin in human endothelial cells.

In certain variations, the deposited low molecular weight organic compound has an enhanced rate of dissolution in comparison to a comparative powder or pellet form of the same deposited low molecular weight organic compound. Thus, a dissolution rate of the deposited low molecular weight organic compound in an aqueous solution (e.g., approximating physiological conditions) is at least ten times greater than a comparative dissolution rate of the comparative powder or pellet form of the deposited low molecular weight organic compound. In certain variations, a dissolution rate of the deposited low molecular weight organic compound in an aqueous solution is at least fifteen times greater, optionally twenty times greater, and optionally thirty times greater than a comparative dissolution rate of the powder or pellet form of the deposited low molecular weight organic compound.

Because biological processes differ for distinct drugs, improving dissolution rate also increases bioavailability, especially for organic compounds where poor dissolution rate is a limitation. Thus, in certain variations, the deposited low molecular weight organic compound has an enhanced bioavailability, for example, an amount and/or rate that the organic compound is absorbed into a living organism or system, as compared to a comparative powder or pellet form of the same low molecular weight organic active ingredient. In certain variations, a bioavailability is enhanced, whether measured by an amount or a rate of uptake of the compound in a living organism or system. Such organisms or living systems may include by way of non-limiting limitation animals, such as mammals like humans and companion animals, plants, bacteria, prokaryotic cells, eukaryotic cells, and the like. In certain examples, bioavailability for a low molecular weight organic active ingredient compound can be increased when it is in the deposited solid form by at least about 10% greater than a comparative bioavailability of the comparative powder or pellet form of the low molecular weight organic active ingredient. The bioavailability may be increased by at least about 20%, optionally at least about 30%, optionally at least about 40%, optionally at least about 50%, optionally at least about 60%, optionally at least about 70%, optionally at least about 80%, optionally at least about 90%, and in certain variations, greater than about 100% of an increase in bioavailability when the low molecular weight organic active ingredient compound is deposited by the methods of the present disclosure as compared to a conventional powder or pellet form of the low molecular weight organic active ingredient compound.

In certain aspects, a solid film having a high surface area morphology can be formed by a modified organic vapor jet printing (OVJP) process, which eliminates the need for organic solvents and improves dissolution rates for small molecular-based organic materials, like APIs. The organic compound(s) that may be deposited by the OVJP process have relatively low molecular weights and thus are considered to be low molecular weight organic compounds. OVJP processes utilize a carrier gas (e.g., nitrogen) to transport sublimated organic vapor towards a cooled substrate or other target in the form of a focused gas jet. The OVJP process enables scalable patterning of relatively small molecular materials.

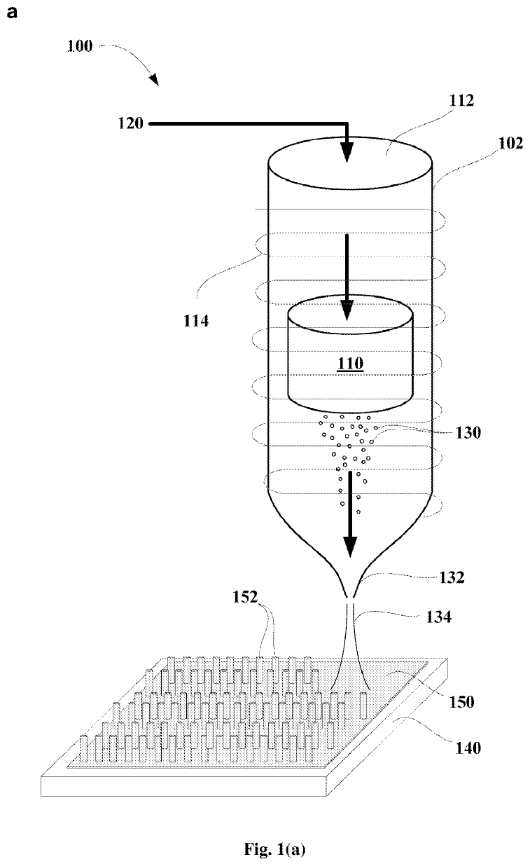

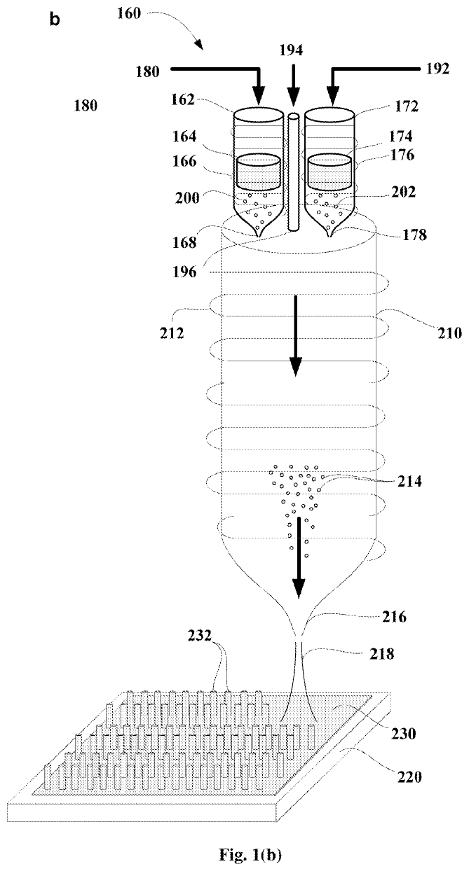

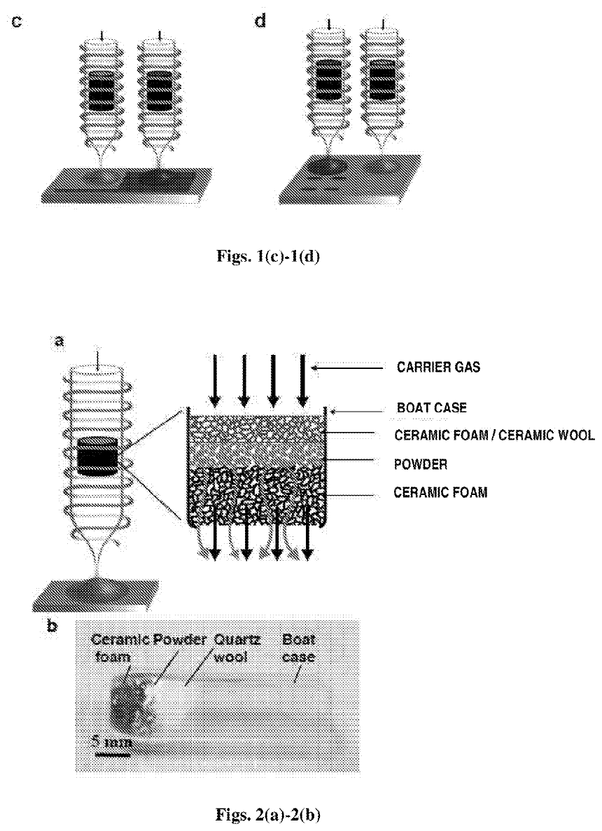

Thus, in certain aspects, an OVJP deposition method is conducted with an OVJP system 100 like that shown in FIG. 1(a). A cylindrical reactor 102 contains a source 110 of the low molecular weight organic compound. The source 110 is in a solid form of the low molecular weight organic compound (e.g., a powder or a pressed pellet). The source 110 may hold or contain the low molecular weight organic compound, for example, as a porous material having the low molecular weight organic compound distributed within pores. The reactor 102 has an inlet 112 in which an inert carrier gas stream 120 enters. A heater 114 is disposed about the exterior or may be otherwise integrated into the reactor 102. A material in the evaporation source 110 is sublimed or evaporated and carried by the inert carrier gas 120. The method thus comprises entraining a low molecular weight organic compound in an inert carrier gas stream 120 by heating the source 110 to sublimate or evaporate the low molecular weight organic compound 130, so that it is a vapor form and entrained in the inert carrier gas stream 120. The entraining can occur by passing the inert carrier gas stream 120 over, by, or through the source 120. Controllable system parameters include carrier gas rate (sccm), evaporation source temperature (° C.), and substrate temperature (° C.). As shown in FIG. 1(a), the low molecular weight organic compound 130 in the inert carrier gas stream 120 is directed through a nozzle 132 in a focused jetted stream 134 towards a cooled target 140. The nozzle 132 is translated above the substrate via xyz motion controllers, enabling printing of any desired deposit pattern.

The cooled target 140 may be a solid or a liquid. The cooled target 140 may be a substrate formed of a material like glass, metals, siloxanes, polymers, hydrogels, organogels, natural fibers, synthetic fibers, and any combinations thereof. As will be described further below, the cooled target 140 may be a microneedle, medical equipment, an implant, a film, a gel, a patch, a dressing, a fabric, a bandage, a sponge, a stent, a contact lens, a subretinal implant prosthesis, dentures, braces, a wearable device, a bracelet, and combinations thereof. When the cooled target 140 is a liquid, it may be a polar or non-polar liquid, including aqueous liquids. The liquid may comprise one or more solvents.

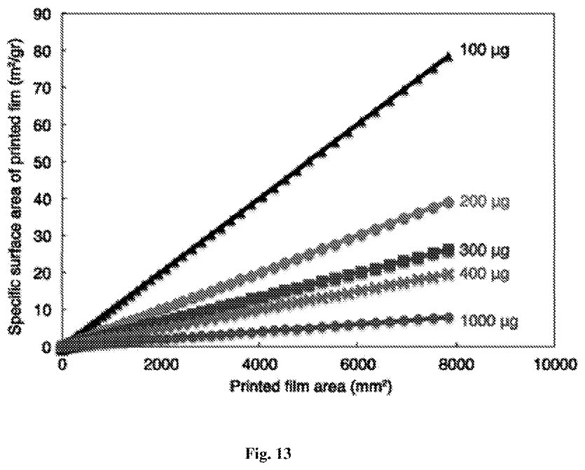

The method further includes condensing the low molecular weight organic compound 130 as it contacts the cooled target 140 on one or more discrete regions. In this manner, the surface of the cooled target 140 may be selectively patterned by directing the jetted stream 134 towards desired regions (or the surface may be temporarily masked). In the variation shown in FIG. 1(a), the one or more discrete regions of the surface of the cooled target 140 are continuous and the condensed low molecular weight organic compound forms a solid film 150 on the surface of the cooled target 140. In certain aspects, the condensed low molecular weight organic compound deposited by OVJP onto the one or more discrete regions of the cooled target 140 may have a loading density of greater than or equal to about 1×10⁻⁴ g/cm² to less than or equal to about 1 g/cm². In certain variations, a specific surface area of the condensed low molecular weight organic compound on the cooled target 140 surface is greater than or equal to about 0.001 m²/g to less than or equal to about 1000 m²/g. FIG. 13 shows a chart of specific film surface area as a function of deposited film area for different printed films weights (of 100 μg, 200 μg, 300 μg, 400 μg, and 1000 μg). The specific surface areas of deposited films are higher for the samples with smaller masses and the specific surface areas are reduced with greater mass. Surface area increases with increasing printed film areas. When nanoparticles are grown on a deposited film, surface area will be enhanced further (about 2 times to 10 times, depending on particle shape and size). For a comparison, powdered organic particles are usually of a size of 1 μm to 100 μm, with surface area 0.1 m²/g to 1 m²/g. Therefore, the enhancement in surface area can be orders of magnitude greater, depending on printed area (as shown in the plot in FIG. 13).

Thicknesses may vary depending on the amount of time that the jetted stream 134 is directed at a particular area of the cooled target 140 surface where the condensed low molecular weight organic compound condenses. In certain variations, when an average thickness of the solid film 150 of condensed low molecular weight organic compound in the one or more discrete regions is less than or equal to about 300 nm, an average surface roughness (Rₐ) of the surface profile (the two-dimensional profile of the surface taken perpendicular to the lay, if any) is less than or equal to about 100 nm. As noted above, for solid films 150 with a thickness of 200±100 nm, the films are generally flat with a surface roughness of less than about 100 nm. Starting with a thickness of around 200±100 nm, undulations occur in a solid film 150, which further produces and define a plurality of nanostructures 152. In this manner, the surface of the solid film 150 is nanostructured.

Where an average thickness of the solid film is greater than or equal to about 300 nm, an average surface roughness (Rₐ) may be greater than or equal to about 100 nm. Further, where an average thickness of the solid film 150 is greater than or equal to about 300 nm, the condensed low molecular weight organic compound may define a nanostructured surface having the plurality of nanostructures 152, which may have a major dimension (e.g., a largest dimension, as shown a length of nanorods or nanocylinders) of greater than or equal to about 5 nm to less than or equal to about 10 μm.

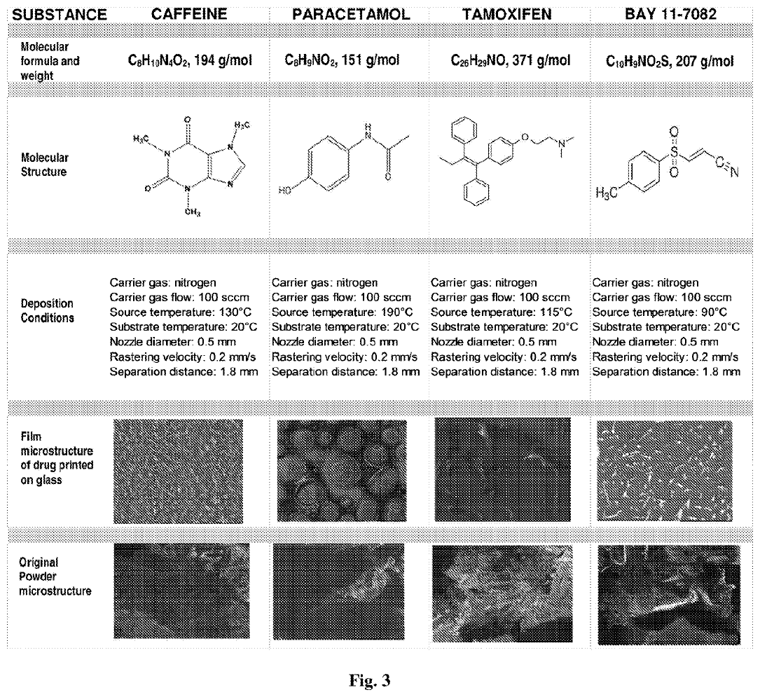

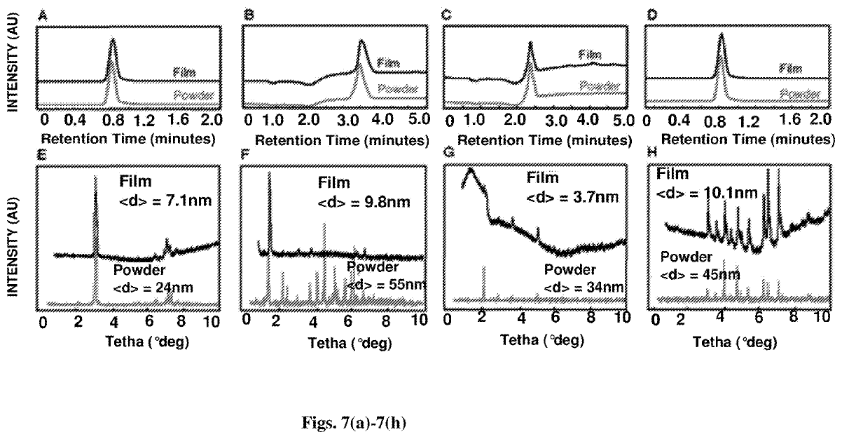

Depending on the OVJP conditions used and the chemistry of the condensed low molecular weight organic compound, the nanostructures 152 may have different shapes. See for example, FIGS. 3 and 7(a)-7(h). In certain aspects, the plurality of nanostructures 152 has a shape selected from the group consisting of: needles, tubes, rods, or cylinders, platelets, round particles, droplets, fronds, tree-like structures, fractals, hemispheres, puddles, interconnected puddles, islands, interconnected islands, and combinations thereof.

While the solid film 150 may have any of the purity levels previously described above, in certain variations, the condensed low molecular weight organic compound is present at greater than or equal to about 99.5 mass %.

Two variations of an OVJP apparatus and process are described herein. In one variation, deposition of the organic compound is performed at atmospheric pressure conditions, rather than pulling a moderate vacuum (10⁻³ Torr). Such a process can be conducted in a glove box with appropriate ventilation. In case of oxygen or moisture-sensitive organic compounds, the process can be performed in a glove box with inert gas environment. In other variations, the entraining and directing are conducted at reduced pressure conditions, for example, at greater than or equal to about 0.1 Torr to less than or equal to about 500 Torr.

In other aspects, a parameter of the OVJP process may be adjusted to affect a morphology, a degree of crystallinity, or both the morphology and the degree of crystallinity of the condensed low molecular weight organic compound. The parameter may be selected from the group consisting of: system pressure, flow rate of the inert gas stream, inert gas composition, a temperature of the source, a composition of a target substrate, a surface texture of the target substrate, a temperature of the target substrate, and combinations thereof. The morphology may include the nanostructures formed. The condensed low molecular weight organic compound in the solid film 150 may be amorphous. In other aspects, the condensed low molecular weight organic compound in the solid film 150 is crystalline or polycrystalline. The low molecular weight organic compound may be any of those described previously above.

FIG. 1(b) shows another OVJP system 160 for conducting an OVJP deposition method similar to that shown in FIG. 1(a), expect that two distinct low molecular weight organic compounds are co-deposited. For brevity, unless specifically discussed herein, components that are the same as those in OVJP system 100 in FIG. 1(a) will not be reintroduced or discussed. In the OVJP system 160, a first cylindrical reactor 162 contains a first source 164 of a first low molecular weight organic compound. The first cylindrical reactor 162 also has a heater 166 and a nozzle 168. A second cylindrical reactor 172 contains a second source 174 of a second low molecular weight organic compound. The second cylindrical reactor 172 also has a heater 176 and a nozzle 178. The first and second sources 164, 174 may be like source 110 in FIG. 1(a). A first inert carrier gas stream 182 enters the first cylindrical reactor 162, while a second inert carrier gas stream 192 enters the second cylindrical reactor 172. A third inert carrier gas stream 194 may pass through a conduit 196. The method thus comprises entraining the first low molecular weight organic compound in first inert carrier gas stream 180 in the first cylindrical reactor 162 by heating the first source 164 to sublimate or evaporate the first low molecular weight organic compound 200 so that it is in a vapor form and entrained in the inert carrier gas stream 180. The second low molecular weight organic compound 202 is also entrained in the second inert carrier gas stream 192 in the second cylindrical reactor 172 by heating the second source 174 to sublimate or evaporate the second low molecular weight organic compound 202 so that it is a vapor form and entrained in the second inert carrier gas stream 192. Notably, the first source 164 in the first cylindrical reactor 162 and the second source 174 in the second cylindrical reactor 172 may be heated to distinct temperature ranges for sublimating or evaporating different low molecular weight compounds with distinct thermodynamic properties. The inert carrier gas stream 180 having the entrained first low molecular weight organic compound 200, the second inert carrier gas stream 192 having the entrained second low molecular weight organic compound 202, and the third inert carrier gas stream 194 all enter a main cylindrical reactor 210 that has a heater 212. The three streams including the vapor phase the first low molecular weight organic compound 200 and second low molecular weight organic compound 202 are combined and mixed together to form a mixed stream 214 that exits a nozzle 216 of the main cylindrical reactor 210 to form a jetted stream 218. Like in FIG. 1(a), the jetted stream 218 comprising the first low molecular weight organic compound 200 in vapor phase and second low molecular weight organic compound 202 in vapor phase is directed through nozzle 216 towards a cooled target 220. The cooled target 220 may be like the cooled target 140 in FIG. 1(a).

The method further includes condensing the first low molecular weight organic compound 200 and second low molecular weight organic compound 202 as they contacts the cooled target 220 in one or more discrete regions. In this manner, the surface of the cooled target 220 may be selectively patterned by directing the jetted stream 218 towards desired regions (or the surface may be temporarily masked). In the variation shown in FIG. 1(b), the one or more discrete regions of the surface of the cooled target 220 are continuous and the condensed low molecular weight organic compound forms a solid film 230 on the surface of the cooled target 220. The solid film 230 may have the same properties as described in the context of solid film 150 in FIG. 1(a), except that it is a homogenous mixture of two distinct low molecular weight organic compounds. As shown, the solid film 230 has nanostructures 232 in the form of nanorods or nanocylinders. The solid film 230 comprises any of the purity levels previously described above when considering the cumulative amount of both the first low molecular weight organic compound 200 and the second low molecular weight organic compound 202, in certain variations, the condensed cumulative amount of low molecular weight organic compounds are present at greater than or equal to about 99 mass % and optionally greater than 99.5 mass % in certain variations. As will be appreciated by those of skill in the art, more than two distinct low molecular weight organic compounds may be applied in an OVJP process and system like that shown.

FIG. 1(c) shows another OVJP system for multilayer mode of deposition for distinct low molecular weight organic compounds, where two distinct cylindrical reactors similar to those described in FIG. 1(b) independently jet directly onto the cooled substrate, so that either a first low molecular weight organic compound or a second low molecular weight organic compound condense on one or more select regions of a cooled substrate. Distinct deposited solid films are thus formed on the cooled target. These films may overlap and form a multi-layered system in one or more regions. FIG. 1(d) shows a patterning mode for an OVJP system like that in FIG. 1(c), where the first low molecular weight organic compound or a second low molecular weight organic compound are respectively applied concurrently to discrete regions of the surface, but do not overlap with one another, to form predetermined patterns (e.g., an array of dots). Any patterns can be made by translating the nozzle independently from one another.

Thus, FIGS. 1(a)-1(d) show several schematics of OVJP systems/devices for making films in accordance with certain variations of the present. The methods of fabricating a surface patterned with an organic compound, such as a pharmaceutical active agent, thus may include sublimating or otherwise volatilizing the organic compound contained in a source/target. A single source or target may be used or multiple sources or targets with multiple distinct organic compounds may also be used (with different configurations shown in FIGS. 1(c) and 1(d)). Likewise, multiple devices may be used in parallel. A system may include one source or multiple sources holding heated small molecular medicine in a powder form. An inert carrier gas (e.g., nitrogen, argon or helium) is introduced to the device and directed towards the source/target of organic material. In certain variations, the organic compound is in solid form, for example, in the form of a powder. Heat is also applied within the system (for example, via a heater) so that organic compound is sublimated or volatilized to a gas/fluid phase and carried by the inert carrier gas stream passing by. The carrier gas having entrained gaseous organic compound is then ejected from a nozzle in a form of focused jet and directed towards a substrate that has a controlled temperature (e.g., may be cooled), where the entrained small organic molecules are condensed. The material can be deposited with precise control of amount, with highly controlled weight ranges of 1×10⁻⁴ g/cm² to 0.1 g/cm², by way of example.

Such a method of fabrication is highly controllable. Various parameters may be controlled in such an OVJP system, including: pressure and flow rate, including carrier gas flow rate (sccm), inert carrier gas type, evaporation source temperature (° C.), and substrate composition, substrate surface texture, and substrate temperature (° C.), by way of example. Changes in each of the parameters can affect film morphology (e.g., features type, size, and distribution) and degree of crystallinity. The nozzle is translated above the substrate via xyz motion controllers, enabling printing of the organic material in any pattern, including a wide variety of preselected deposit patterns. The resolution of a pattern formed depends on nozzle geometry, inert gas type and flow conditions. In order to obtain a large area deposit, adjacent lines of the deposit can be printed with one nozzle or with multiple nozzles. This enables scalability of the process with robust process conditions. Further, in certain aspects, such a method desirably eliminates the requirement for liquid solvents, vacuum, or extensive post-processing steps to obtain a desired particle size for one or more organic compounds. Importantly, such an OVJP works without liquid solvents or vacuum, and allows for controlled degree of crystallization in the organic films.

Further, a new evaporation source/target is contemplated by the present disclosure for vapor deposition methods of low molecular weight organic compounds. As shown in FIGS. 2(a)-2(b), a ceramic porous powder holder is provided. The evaporation/volatilization source includes an outer container—made of either thermally or mechanically deformable glass or metal, with a disk made of porous ceramic (e.g., reticulated) foam serving as a powder holder (FIG. 2(a)). The powder of the organic material is covered with either another ceramic foam disk or with ceramic wool (glass/quartz). The porous ceramic foam can comprise oxides, nitrides, carbides, borides, silicides or any combination of thereof, provided the organic material to be deposited does not adversely interact (e.g., chemically decompose) with the foam. The foam is then cut to the needed shape of the container. Due to high thermal and mechanical stability of ceramic foam, the container with the foam can be compression heated to ensure tight positioning of the foam, thus ensuring reproducibility of the process when replacing the powder and preventing powder spillage during the process. One variation of such a source of organic material is shown in FIG. 2(b). The boat case is made of quartz and the foam is made of silicon carbide from Ultramet. The powder to be deposited is organic molecular substance Alq₃, with sublimation point of approximately 300° C.

One or more non-limiting advantages and/or features of the processes according to the present disclosure include: (i) that the method is highly controllable. As noted above, the control parameters include: evaporation source temperature, inert carrier gas type, pressure and flow rate, substrate composition, surface texture and temperature. Changes in each of the parameters can affect film morphology (e.g., features type, size, distribution) and degree of crystallinity; (ii) eliminating the requirement for solvents or extensive post-processing steps to obtain the desired particle size; (iii) enable deposition of a wide range of small molecular organic medicines with molecular weight up to 1000 gr/mole; (iv) that the low molecular weight organic material can be deposited with precise control of amount, up to 1e⁻⁹ grams; (v) the low molecular weight organic material can be printed in any pattern; (vi) the resolution of the pattern depends on nozzle geometry, inert gas type and flow conditions. In order to obtain a large area deposit, adjacent lines of the deposit can be printed with one nozzle or with multiple nozzles. This enables scalability of the process with robust process conditions; (vii) the low molecular weight organic materials can be co-printed as a mixture of multiple compounds; (viii) can be conducted continuously in roll-to-roll manufacturing; (ix) can enable printing a personalized dosage of substance or mixture of substances; and (x) the deposition apparatus can be highly compact, enabling equipment mobility and usage in a modular manner (many nozzles arranged in any way needed), as well as incorporating the system as a manufacturing module.

FIG. 3 shows a variety of examples of printed pharmaceutical materials (e.g., organic compounds) in accordance with the organic vapor jet deposition printing processes of the present teachings. All materials are deposited while rastering the nozzle at a velocity of 0.2 mm/s, while the adjacent lines are 0.2 mm apart from one another. Nozzle tip diameter in all tests is 0.5 mm. All depositions are performed at atmospheric pressure in inert nitrogen environment (<1 ppm O₂ and H₂O). Electron micrographs are included in the table of FIG. 3, indicating refinement of original powder microstructure due to the deposition/printing process. While not shown, X-ray diffraction patterns further demonstrate that the crystal structure of the films is comparable to the crystal structure of the original source material, indicating that the crystal structure is unaltered during deposition. HPLC results of initial powder and the films indicate high material purity after the deposition.

In other aspects, the present disclosure contemplates a method for rapid dissolution of low molecular weight organic compounds. Small molecular organic vapor compounds may be jetted directly into liquids. In certain aspects, the liquid may be an aqueous solution, demonstrating how precise drug concentrations can be rapidly reached, without the need for additional solvents and/or powder preparation. Solutions of small molecular organic compounds are used extensively in many industries: food, cosmetics/perfume, pharmaceuticals, printing and paints. As background, conventionally to achieve a given concentration of organic solute in original powder form, the required amount of powder is immersed directly in the solvent and is dissolved until all powder particles are separated into solvated molecules. This process is especially challenging for low solubility substances, where dissolution rate is very slow. To enhance dissolution rates, powder particle size is reduced (via milling or other methods), and solution is usually heated. This approach can be both time and energy consuming, as well as potentially damaging to the solvent.

An additional drawback of the conventional technique of direct immersion of powder solute in the solvent is when the actual needed concentration of a compound or solution volume is very low. For instance, if a desired concentration is on the order of micromolar, and volume needed is 10 ml, the weight needed for a 200 g/mole material would be on the order of micrograms. This weight is not feasible to measure accurately for a precursor powder, therefore a higher concentration of solution is made with subsequent dilution with additional amount of solvent. This process is undesirable from both economical and safety standpoint (when dealing with organic solvent).

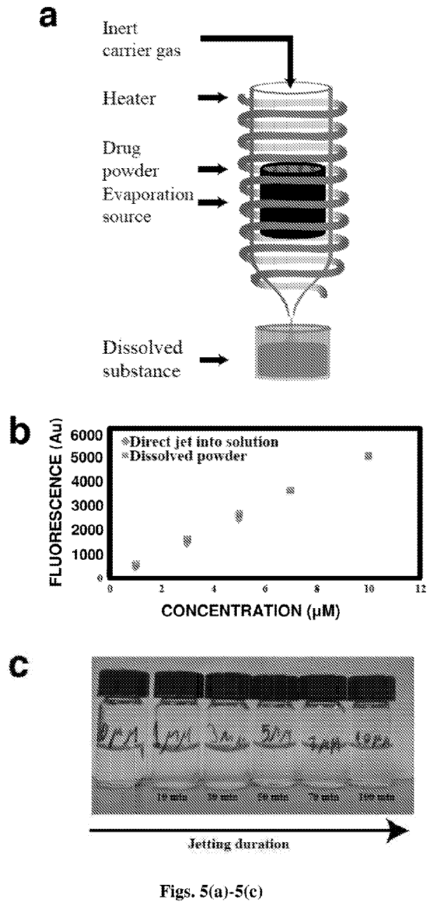

A method for rapid dissolution of low molecular weight organic compounds is also provided that includes passing a gas stream comprising an inert gas past a heated source of the low molecular weight organic compound(s), as shown in FIG. 5(a). The low molecular weight organic compound is volatilized and entrained in the gas stream. Then, the low molecular weight organic compound is jetted into a liquid comprising one or more solvents by passing the gas stream through a nozzle towards the liquid. In this manner, the deposited low molecular weight organic compound is desirably dissolved in the liquid. The liquid may be a polar or non-polar liquid, including aqueous liquids comprising water or miscible with water. The liquid may thus comprise one or more solvents.

The heated source may comprise a porous ceramic holder comprising the low molecular weight organic compound that receives heat transferred from a heater, such as the porous ceramic holder described above in the context of FIGS. 2(a)-2(b). In certain aspects, the heated source has a temperature of greater than or equal to about 250° C. and the liquid is at ambient temperature. The nozzle may be greater than or equal to about 15 mm to less than or equal to about 25 mm from a surface of the liquid. The inert gas may be nitrogen. After dissolution, a concentration of the low molecular weight organic compound is optionally greater than or equal to about 1×10⁻¹¹ mol/L to less than or equal to about 20 mol/L. In certain variations, an amount of low molecular weight organic compound deposited is less than or equal to about 100 μg. In other variations, a volume of the liquid into which the gas stream comprising the low molecular weight organic compound is deposited/jetted is less than or equal to about 100 ml. The depositing is conducted for greater than or equal to about 1 minute to less than or equal to about 120 minutes. The low molecular weight organic compound may be any of those previously described above, by way of non-limiting example, the low molecular weight organic compound may be selected from the group consisting of: caffeine, (E)-3-(4-Methylphenylsulfonyl) propenenitrile, fluorescein, paracetamol, ibuprofen, tamoxifen, and combinations thereof.

Thus, the present disclosure contemplates a new dissolution method and an apparatus for conducting such a process, as shown in FIGS. 5(a)-5(c). The apparatus shown in FIG. 5(a) includes a heated organic powder evaporation source in a ceramic tube, similar to those described previously above. The temperature of the source is high enough to cause evaporation/volatilization/sublimation of the organic material. An inert carrier gas is flowing through the powder, picking up and volatilizing/evaporating molecules and delivering them into a solution. Using this method, a precise and controlled amount of organic material can be jetted into solution with sub-micromolar concentrations. An example of fluorescein (molecular weight 332 g/mole) jetted into phosphate buffer saline solution with micromolar concentrations is shown in FIG. 5(b). In FIG. 5(c), a concentration of the fluorescein in the solution is shown to vary by jetting duration (e.g., from 0 minutes to 100 minutes of jetting). Concentration was measured by fluorescence spectroscopy calibrated with dissolved fluorescein powder.

In certain aspects, the present disclosure thus contemplates a solid film comprising greater than or equal to about 99 mass % of a deposited low molecular weight organic active ingredient compound having a molecular weight of less than or equal to about 1,000 g/mol. For example, the deposited low molecular weight organic compound may have a molecular weight of greater than or equal to about 100 g/mol to less than or equal to about 900 g/mol. The low molecular weight organic active ingredient compound is preferably a pharmaceutical active or a new chemical entity. The low molecular weight organic active ingredient is any of the low molecular weight compounds described above. By way of example, the deposited low molecular weight organic active ingredient compound may be selected from the group consisting of: anti-proliferative agents; anti-rejection drugs; anti-thrombotic agents; anti-coagulants; antioxidants; free radical scavengers; nucleic acids; saccharides; sugars; nutrients; hormones; cytotoxin; hormonal agonists; hormonal antagonists; inhibitors of hormone biosynthesis and processing; antigestagens; antiandrogens; anti-inflammatory agents; non-steroidal anti-inflammatory agents (NSAIDs); antimicrobial agents; antiviral agents; antifungal agents; antibiotics; chemotherapy agents; antineoplastic/anti-miotic agents; anesthetic, analgesic or pain-killing agents; antipyretic agents, prostaglandin inhibitors; platelet inhibitors; DNA de-methylating agents; cholesterol-lowering agents; vasodilating agents; endogenous vasoactive interference agents; angiogenic substances; cardiac failure active ingredients; targeting toxin agents; and combinations thereof. In certain variations, the deposited low molecular weight organic active ingredient compound is selected from the group consisting of: caffeine, (E)-3-(4-Methylphenylsulfonyl)-2-propenenitrile, fluorescein, paracetamol, ibuprofen, tamoxifen, and combinations thereof.

In certain aspects, the solid film has a specific surface area of the solid film that is greater than or equal to about 0.001 m²/g to less than or equal to about 1,000 m²/g. In certain variations, the deposited low molecular weight organic active ingredient compound in the solid film is amorphous. The solid film may further define particles having an average particle size of greater than or equal to about 2 nm to less than or equal to about 200 nm. Where the solid film is amorphous, the deposited low molecular weight organic active ingredient compound in the solid film is stable for greater than or equal to about 1 month, optionally greater than or equal to about 2 months, optionally greater than or equal to about 3 months, optionally greater than or equal to about 6 months, optionally greater than or equal to about 9 months, and in certain variations, optionally greater than or equal to about 1 year.

In other variations, the deposited low molecular weight organic active ingredient compound in the solid film is crystalline or polycrystalline. An average crystal size may be greater than or equal to about 2 nm to less than or equal to about 200 nm. An average thickness of the solid film may be less than or equal to about 300 nm and an average surface roughness (Rₐ) of the solid film is less than or equal to about 100 nm.

In other variations, an average thickness of the solid film is greater than or equal to about 300 nm. An average surface roughness (Rₐ) is greater than or equal to about 100 nm. The film having such a thickness defines a nanostructured surface comprising a plurality of nanostructures having a major dimension of greater than or equal to about 5 nm to less than or equal to about 10 μm. In such an embodiment, the plurality of nanostructures may have a shape selected from the group consisting of: needles, tubes, rods, platelets, round particles, droplets, fronds, tree-like structures, fractals, hemispheres, puddles, interconnected puddles, islands, interconnected islands, and combinations thereof.

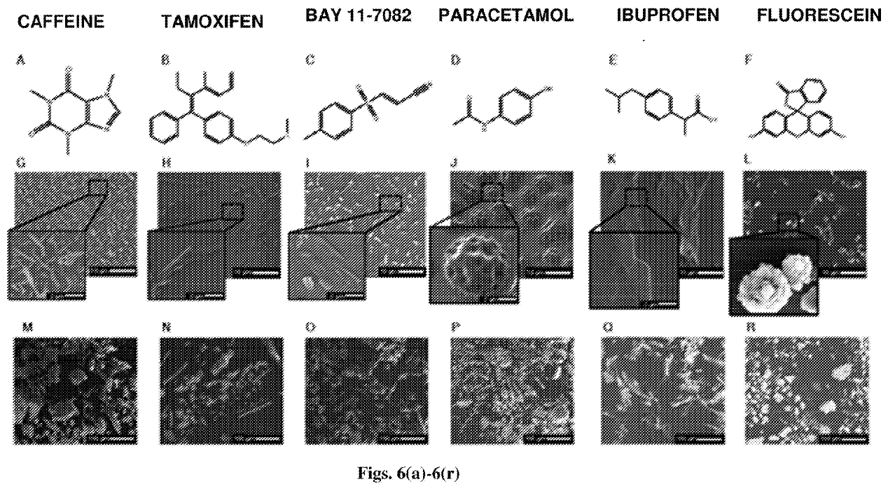

FIGS. 6(a)-6(r) shows surface morphology of solid printed films for caffeine, tamoxifen, BAY 11-7082, paracetamol, ibuprofen, and fluorescein deposited with OVJP. FIGS. 6(a)-6(f) show chemical structures of the compounds. FIGS. 6(g)-6(l) show deposited film morphologies after jetting in accordance with the certain aspects of the present teachings. FIGS. 6(m)-6(r) show original microstructure of powders of the compounds. All materials are deposited while rastering the nozzle at a velocity of 0.2 mm/s, while the adjacent lines were 0.2 mm apart from one another. Nozzle tip diameter in all tests is 0.5 mm. All depositions are performed at atmospheric pressure in inert nitrogen environment (<1 ppm O₂ and H₂O). Electron micrographs indicate refinement of original powder microstructure due to the printing process.

Table 1 lists the OVJP deposition conditions of the printed films.

TABLE 1

| | Process parameter | | | |
|---|---|---|---|---|
| | | | | Target |
| | | Carrier | Source | Substrate |
| | Carrier | gas rate | temperature | temperature |
| Material | gas type | (sccm) | (° C.) | (° C.) |
| Fluorescein | Nitrogen | 200 | 300 | 20 |
| Caffeine | Nitrogen | 100 | 130 | 20 |
| Tamoxifen | Nitrogen | 100 | 115 | 20 |
| BAY 11-7082 | Nitrogen | 100 | 90 | 20 |
| Paracetamol | Nitrogen | 100 | 190 | 20 |
| Ibuprofen | Nitrogen | 150 | 75 | 20 |

Source temperature is determined via thermogravimetry and tuned to obtain local deposition rate of approximately 0.5 μg/min. The temperature range and carrier gas rate can change depending on system size and configuration.

In certain embodiments, the solid film may comprise a deposited low molecular weight organic compound comprising caffeine. The plurality of nanostructures has a needle shape or a tube shape. An average diameter of the plurality of nanostructures is greater than or equal to about 5 nm to less than or equal to about 10 μm and an average length of greater than or equal to about 5 nm to less than or equal to about 100 μm. FIG. 6(a) shows the chemical structure; FIG. 6(g) shows a micrograph of the morphology of the deposited film having nanostructures in the form of a needle or tube shape; while FIG. 6(m) shows the morphology of conventional powder.

In certain other embodiments, the solid film may comprise a deposited low molecular weight organic compound comprising (E)-3-(4-Methylphenylsulfonyl)-2-propenenitrile (BAY 11-7082). The plurality of nanostructures has a platelet shape, where an average height of the plurality of nanostructures is greater than or equal to about 10 nm to less than or equal to about 10 μm. An average width of the plurality of nanostructures is greater than or equal to about 5 nm to less than or equal to about 10 μm. An average length of greater than or equal to about 5 nm to less than or equal to about 100 μm. FIG. 6(c) shows the chemical structure, FIG. 6(i) shows a micrograph of the morphology of the deposited film having nanostructures in the form of platelets, while FIG. 6(o) shows the morphology of conventional powder.

In yet other embodiments, the solid film may comprise a deposited low molecular weight organic compound comprising fluorescein. The plurality of nanostructures has a round shape. An average radius of the plurality of nanostructures is greater than or equal to about 5 nm to less than or equal to about 10 μm. FIG. 6(f) shows the chemical structure, FIG. 6(l) shows a micrograph of the morphology of the deposited film having nanostructures in the form of round nanostructures, while FIG. 6(r) shows the morphology of conventional powder.

In yet further embodiments, the solid film may comprise a deposited low molecular weight organic compound comprising paracetamol. The plurality of nanostructures has a shape selected from the group consisting of: droplet, hemisphere, puddle, interconnected puddle, island, interconnected island, and combinations thereof, wherein an average major dimension of the plurality of nanostructures is greater than or equal to about 5 nm to less than or equal to about 20 μm. FIG. 6(d) shows the chemical structure, FIG. 6(j) shows a micrograph of the morphology of the deposited film having nanostructures in the form of a droplet shape, while FIG. 6(p) shows the morphology of conventional powder.

FIG. 6(b) shows the chemical structure of tamoxifen. FIG. 6(h) shows a micrograph of the morphology of the deposited film having nanostructures in the form of continuous platelet-like shapes, while FIG. 6(n) shows the morphology of conventional powder.

FIG. 6(e) shows the chemical structure of ibuprofen. FIG. 6(k) shows a micrograph of the morphology of the deposited film having nanostructures in a form of droplet-like, yet solid aggregates, while FIG. 6(q) shows the morphology of conventional powder.

X-ray diffraction patterns (FIGS. 7(e)-7(h)) demonstrate that the crystal structure of the films is comparable to the crystal structure of the original source material, indicating that the crystal structure was unaltered during deposition. Crystal size of deposited compounds is substantially refined, from tens of nanometers in powder to several nanometers in a film. UPLC results of initial powder and the films indicate high material purity after the deposition are shown in FIGS. 7(a)-7(d).

In other aspects, the deposited low molecular weight organic compound according to the present teachings has an enhanced rate of dissolution as compared to a comparative powder or pellet form of the low molecular weight organic active ingredient. A dissolution rate of the deposited low molecular weight organic active ingredient compound in the solid film in an aqueous solution is at least ten times greater than a comparative dissolution rate of the comparative powder or pellet form of the low molecular weight organic active ingredient. The dissolution rate improvement may be any of those previously discussed above.

Dissolution process in a finite volume can be described by Noyes-Whitney (Equation 1), where C— is solute concentration, t—is time, D—is diffusion coefficient in the solvent, V— is solvent volume, δ—is boundary layer thickness, Cs—is solubility in a given solvent and A is a surface area of the solute:

$$\frac{d C}{d t} = \frac{D A ( t )}{V δ ( t )} ( {C}_{s} - C ) \qquad ( 1 )$$

When comparing dissolution from powder form and film form for the same material and same crystal structure, D, V, Cs are constant and initial dissolution rate will be proportional to A/δ. δ and A are constant for dissolution from film since area of the film is not changing during dissolution. In a powder, δ and A are changing since particles size and shape is changing. In addition, particles have a tendency to agglomeration during a dissolution, which does not occur when dissolving from a film form. Therefore only the initial rate can be compared between powder and a film.

The degree of enhancement in dissolution rate will be similar to degree of enhancement in surface area. In specific and non-limiting examples, fluorescein in deionized water has an initial dissolution rate of 25 μg of powder of 8.9e⁻⁵ μg ml⁻¹ sec⁻¹, while printed film is 1.61e⁻³ μg ml⁻¹ sec⁻¹, which is 18 times higher. Film surface area is 6.4e⁻⁵ m², while powder surface area is 3.6e⁻⁶ m², 17 times higher.

Ibuprofen in buffer HCl 1.3 has an initial dissolution rate for 30 μg of powder of 0.0004 μg ml⁻¹ sec⁻¹, while of printed film is 0.04 μg ml⁻¹ sec⁻¹, about 10 times higher. Film surface area is 6.4e⁻⁵ m², while powder surface area is 1.5e⁻⁵ m², about 5 times higher.

Tamoxifen in buffer acetate 4.9 has an initial dissolution rate for 30 μg of powder is 2 e⁻⁴ μg ml⁻¹ sec⁻¹, while that of printed film is 2 e⁻³ μg ml⁻¹ sec⁻¹, 10 times higher. Film surface area is 6.4e⁻⁵ m², while powder surface area is 9e⁻⁶ m², 7 times higher. Notably, these are only illustrative examples, but surface area of film is related to printed surface area, which is not limited in accordance with the present teachings.

Likewise, the deposited low molecular weight organic compound according to the present teachings has an enhanced bioavailability as compared to a comparative powder or pellet form of the low molecular weight organic active ingredient. Enhanced bioavailability is related to dissolution rate enhancement. A bioavailability of the deposited low molecular weight organic active ingredient compound in the solid film is at least about 10% greater than a comparative bioavailability of the comparative powder or pellet form of the low molecular weight organic active ingredient. The bioavailability enhancement levels may be any of those previously specified above.

In certain aspects, the solid film is substantially free of any binders or impurities. A solid film that is substantially free of binders or impurities has less than or equal to about 0.5% by weight, optionally less than or equal to about 0.1% by weight, and in certain preferred aspects, 0% by weight of the undesired binders or impurities present in the solid film composition. In certain variations, the solid film comprises greater than or equal to about 99.5 mass % of the deposited low molecular weight organic active ingredient compound; however, any of the purity levels discussed above may likewise be achieved in the solid film.

In certain aspects, the deposited low molecular weight organic compound on the surface is crystalline or polycrystalline. In other aspects, the deposited low molecular weight organic compound is amorphous. In this manner, substantially pure molecular medicinal films are fabricated that may have high surface area morphologies. The deposited low molecular weight organic compound exhibits enhanced solubility and bioavailability.

In other variations, the present disclosure contemplates a solid film comprising multiple deposited low molecular weight organic active ingredient compounds each having a molecular weight of less than or equal to about 1,000 g/mol. The low molecular weight organic active ingredient compounds are preferably a pharmaceutical active or a new chemical entity. The low molecular weight organic active ingredient compounds are any of the low molecular weight compounds described above. A collective amount of the multiple low molecular weight organic active ingredient compounds may be greater than or equal to about 99 mass % in the solid film. The solid films may have any of the compositions or features described just above, which will not be repeated herein for brevity.

In yet other variations, an article comprises a solid deposited film comprising a pharmaceutical composition comprising at least one low molecular weight organic compound having a molecular weight of less than or equal to about 1,000 g/mol. The solid deposited films may have any of the composition or features described above, which will not be repeated herein for brevity.

In certain other variations, an article is provided that includes a surface of a solid substrate having one or more discrete regions patterned with a deposited low molecular weight organic compound having a molecular weight of less than or equal to about 1,000 g/mol. The low molecular weight organic compound is any of the low molecular weight compounds described above. The deposited low molecular weight organic compound is present at greater than or equal to about 99 mass % in the one or more discrete regions. In certain aspects, the one or more discrete regions of the surface are continuous and the deposited solid low molecular weight organic compound forms a solid film on the surface of the pharmaceutically acceptable substrate. Any of the solid films described above, including any of the compositions or features described above, may be disposed on a surface of a solid substrate. The deposited film may be applied to a variety of solid substrates having any type of substrate geometry, including flat substrates, microneedles, spheres, tubes, curved surfaces, meshes, fabrics, and combinations thereof.

In certain other variations, an article is provided that includes a surface of a solid substrate having one or more discrete regions patterned with multiple deposited low molecular weight organic compounds each having a molecular weight of less than or equal to about 1,000 g/mol. The low molecular weight organic compounds are any of the low molecular weight compounds described above. The multiple deposited low molecular weight organic compounds are cumulatively present at greater than or equal to about 99 mass % in the one or more discrete regions. Thus, any of the solid films described above may be disposed on a surface of a solid substrate. Further, the solid substrate may be as described just above.

In yet other aspects, the present disclosure provides an article comprising a pharmaceutically acceptable substrate defining a surface. The materials selected for the substrate are preferably pharmaceutically acceptable or biocompatible, in other words, substantially non-toxic to cells and tissue of living organisms. Pharmaceutically acceptable materials may be those which are suitable for use in contact with the tissues of humans and other animals without resulting in excessive toxicity, irritation, allergic response, or other problems or complications, commensurate with a reasonable benefit/risk ratio. The article also includes a deposited solid low molecular weight pharmaceutical active ingredient having a molecular weight of less than or equal to about 1,000 g/mol. A pharmaceutical active ingredient is a drug or other compound operable for the prevention or treatment of a condition or disorder in a human or other animal, the prevention or treatment of a physiological disorder or condition, or to provide a benefit that outweighs potential detrimental impact in a conventional risk-benefit assessment. The low molecular weight organic active ingredient may be any of those described above. Thus, the articles and compositions of the present disclosure may be used for the treatment or prevention of systemic disorders, such as cancer, autoimmune diseases, cardiovascular disease, stroke, diabetes, severe respiratory infection, inflammation, pain control, and the like.

The deposited solid low molecular weight pharmaceutical active ingredient is present at greater than or equal to about 99 mass % in one or more discrete regions on the surface of the pharmaceutically acceptable substrate. The one or more discrete regions of the surface are continuous and the deposited solid low molecular weight pharmaceutical active ingredient forms a solid film on the surface of the pharmaceutically acceptable substrate. Thus, any of the solid films described above having a low molecular weight pharmaceutical active ingredient may be disposed on a surface of a solid substrate.

In certain aspects, the pharmaceutically acceptable substrate is biodegradable. By biodegradable, it is meant that the materials forming the substrate dissolve or erode upon exposure to a solvent comprising a high concentration of water, such as serum, growth or culture media, blood, bodily fluids, or saliva. In some variations, a substrate may disintegrate into small pieces or may disintegrate to collectively form a colloid or gel. In certain variations, the pharmaceutically acceptable substrate comprises a pharmaceutically acceptable material selected from the group consisting of: glass, metals, siloxanes, polymers, hydrogels, organogels, organic materials, natural fibers, synthetic fibers, ceramic, biological tissue, and combinations thereof. In other variations, the pharmaceutically acceptable material is selected from the group consisting of: glass, metals, siloxanes, polymers, hydrogels, organogels, natural fibers, synthetic fibers, and combinations thereof. The deposited solid low molecular weight pharmaceutical active ingredient can be formed on any type of substrate geometry, including flat substrates, microneedles, spheres, tubes, curved surfaces, meshes, and the like. Further, the substrate can be of any size. In certain non-limiting variations, the pharmaceutically acceptable substrate is selected from the group consisting of: a microneedle, medical equipment, an implant, a film, such as a dissolvable film or a film having a removable backing, a gel, a patch, a dressing like a gauze, a non-adhesive mesh, a bandage, a membrane, a foil, a foam, or a tissue adhesive, a fabric, such as a woven, nonwoven, or knitted fabric, a sponge, a stent, a contact lens, a subretinal implant prosthesis, dentures, braces, a wearable device, a bracelet, and combinations thereof.

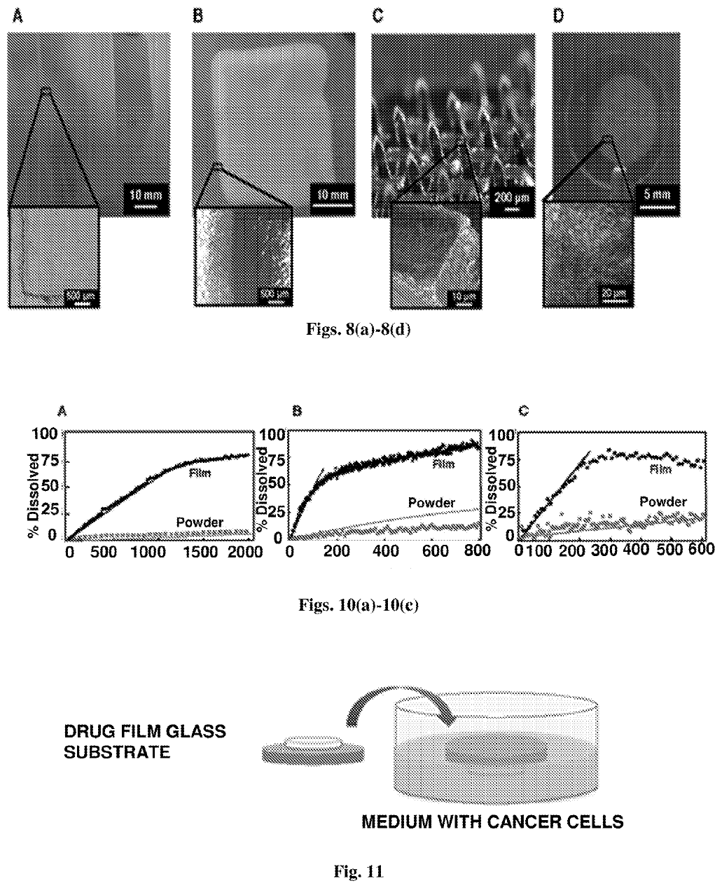

FIGS. 8(a)-8(d) demonstrate examples of different coating modes on different substrates. The low molecular weight compound fluorescein is patterned onto an acrylic polymer TEGADERM™ patch sold by 3M™ (FIG. 8(a)) and pullulan-based LISTERINE® films (FIG. 8(b)), fluorescein deposited onto the tips of stainless steel microneedles (FIG. 8(c)), and tamoxifen deposited onto borosilicate glass slide (FIG. 8(d)).

In other aspects, the present disclosure contemplates an article comprising a solid deposited film comprising a pharmaceutical composition. The pharmaceutical composition comprises at least one low molecular weight organic compound having a molecular weight of less than or equal to about 1,000 g/mol. In certain variations, the pharmaceutical composition further comprises at least one additional deposited compound distinct from the low molecular weight organic compound, so that a plurality of low molecular weight organic compounds are co-deposited to form a solid deposited film. Thus, the pharmaceutical composition may comprise at least two low molecular weight organic compounds. In certain variations, the pharmaceutical composition has at least one low molecular weight organic compound present at greater than or equal to about 99 mass % in the solid deposited film.

The article may be a multilayered stack and the solid deposited film comprising the pharmaceutical composition is a first layer and the multilayered stack comprises a second layer having a distinct chemical composition. The second layer may include a second distinct pharmaceutical composition from pharmaceutical composition in the first layer. In other aspects, the second layer comprises a material that minimizes dissolution rate of the pharmaceutical composition in the first layer. The second layer in other variations may comprise a material having a solubility controlled by the presence of a trigger selected from the group consisting of: light, radiation, magnetism, radio waves, pH of a surrounding medium, and combinations thereof. In this manner, such external forces or triggers can be used to enhance or minimize solubility of the pharmaceutical composition. The pharmaceutical composition may have any of the compounds and attributed previously discussed. Further, the solid deposited film may have any of the features or properties previously discussed.

### Example

OVJP nozzles used are made from quartz tubes of 0.5″ outer diameter with nozzle tip of 0.5 mm internal diameter with 15° C. from nozzle axis. All nozzles used are identical. The inert gas used during deposition is 99.99% pure nitrogen.

The nozzles are cleaned with acetone and isopropanol solvents, dried and wrapped with 36″ gauge heavy insulated tape heater (Omega Engineering, Inc.) with a power density of 8.6 W·in⁻². The heating tape leads are connected to a temperature controller (Digi-Sense Benchtop temperature controller, Cole Palmer Instruments Co.) and a 1/16″ K type thermocouple was used to maintain the temperature of the source. The source comprises about 0.15 g of powder embedded in a porous SiC ceramic foam of 100 DPI and placed in the heated source section of the tube. The gas flow rates are maintained using mass flow controllers (C100 MFC, Sierra Instruments).

The process parameters that are kept constant are: nozzle-substrate separation distance (1.5 mm), substrate temperature (20° C.). The process is performed in glove box purged with 99.99% pure N₂. Thermogravimetry of pharmaceutical substances

In order to determine evaporation temperature of the powders, and subsequently source temperature in the system, thermogravimetry analysis is used. All measurements are performed using a TA Instruments thermogravimetric analyzer (TGA) Q500 system (0.01% accuracy) with nitrogen sample purge flow rate 60 ml/min and balance purge flow rate of 40 ml/min. Heating rate is 5° C./min.

Area deposits are printed by rastering adjacent overlapping lines at distance of 0.2 mm. This distance is determined to allow for homogeneous thickness of deposit for a nozzle of 0.5 mm inner diameter positioned 1.5 mm from substrate surface. Fluorescein films on microneedles are deposited through a flexible mask. The same process can be performed without mask when using nozzle with appropriate printing resolution.

In certain aspects, the OVJP processes conducted in accordance with certain aspects of the present teachings can deliver controlled amounts of various compounds (e.g., caffeine, ibuprofen, doxorubicin, BAY 11-7082) onto various substrates in film form. How the film then dissolves in aqueous solution is further observed. The film dissolution process is monitored using fluorescent substances, such as fluorescein.

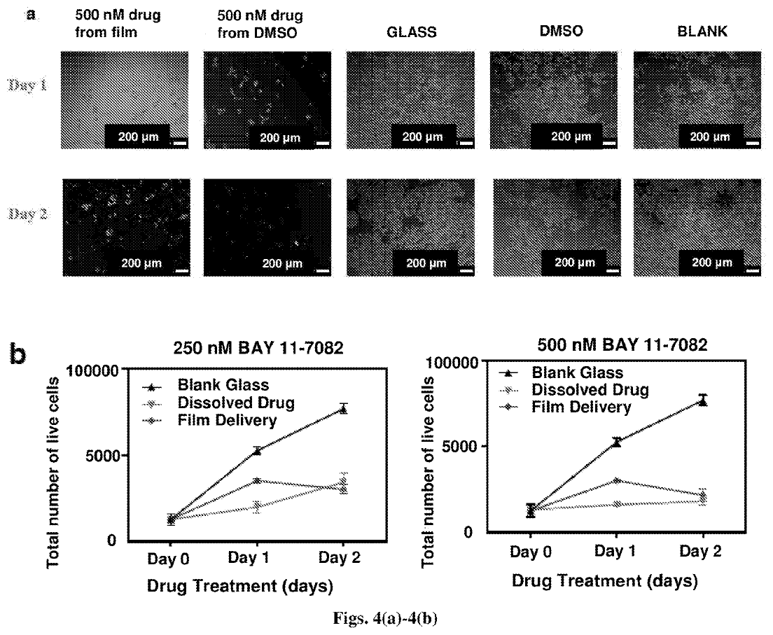

FIGS. 4(a)-4(b) show an example of printed pharmaceutical film with tested biological efficacy of a deposited organic compound, BAY 11-7082 (CAS 19542-67-7). The film was deposited at conditions indicated in FIG. 3. The printed films of BAY 11-7082 are tested by applying OVCAR3 cells solution directly onto the film, as compared to the BAY 11-7082 drug in powder form dissolved in DMSO. No significant difference in efficacy was observed, indicating that the film has enhanced solubility properties.

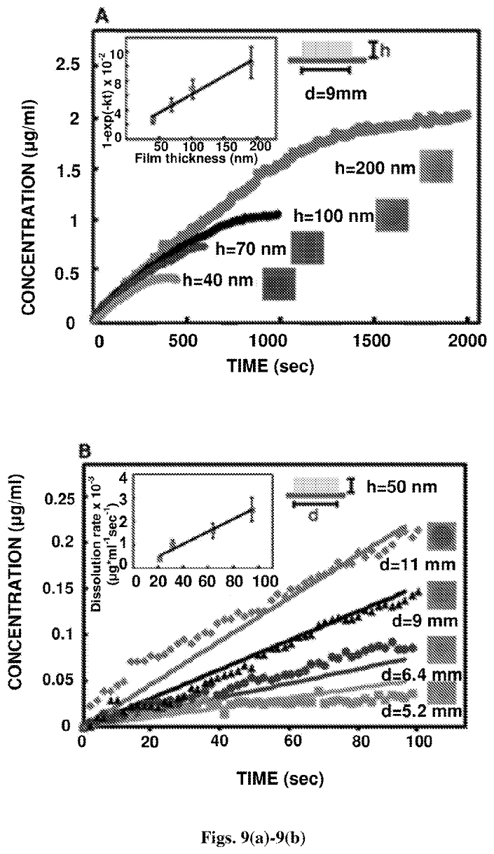

FIGS. 9(a)-9(b) demonstrate how dissolution (or release) rate of films can be controlled via film patterning. In a first case, a deposited film thickness is changed, while film area remained constant (FIG. 9(a)). Here dissolution rate is not changing and precise final concentration is achieved. Concentration—dissolution time dependence is shown in the inset of FIG. 9(a). FIG. 9(b) demonstrates dissolution from films with different deposited areas. Here dissolution rate is proportional to film area. In both cases the dependence is well predicted by Noyes-Whitney theory.

Enhancement in dissolution rates of pharmaceutical films printed from vapor phase in accordance with certain aspects of the present teachings versus pharmaceuticals in powder form are shown in FIGS. 10(a)-10(c). In order to compare film form versus original powder dissolution behavior, loose powders with same weight as films are introduced into 10 ml solution without any prior treatment and stirred using stirring rod with same shape and diameter as one that is used for films. All experiments are performed at temperature 19±1° C.

In case of dissolution from film, the exposed dissolving area and boundary layer thickness are not changing and solution to the Equation (1) is Equation (2):

$$C = {C}_{s} ( 1 - {exp}^{- \frac{D A t}{V δ}} ) \qquad ( 2 )$$

In case of sink condition, C<<Cs, the dissolution rate is essentially constant and therefore can be precisely controlled by film area. Dissolution process in powder form is less controllable than in film form. As opposed to film form, in case of powder, the active dissolution area is changing during the process and will be affected by change in particle size and shape, wettability and tendency to agglomerate. Simplified solution to equation 1 is described by Hixson and Crowell model (Equation (3)), where N—number of powder particles, Mp₀- particles average initial weight, ρ—solute material density.

$$C = \frac{N}{V} [ M {p}_{0} - ( {Mp}_{0}^{1 / 3} - {( {( \frac{4 π}{3 {ρ}^{2}} )}^{1 / 3} \frac{D {C}_{s}}{3 δ} )}^{3} t ) ] \qquad ( 3 )$$

The model does not include effects like change in particle shape, boundary layer thickness, tendency to agglomeration, wettability and assumes rounded particle shape, which is not common shape in crystalline organic solids. Powder micronization techniques that are used to increase the dissolution rate, are limited by processing conditions and powder agglomeration. When depositing a drug in a film form these limitations essentially do not exist. The deposited film can be as thin as one monolayer of a material.

Dissolution behavior in film and powder form is studied here in three poorly soluble materials—fluorescein in deionized water, ibuprofen in aqueous hydrochloride (HCl) buffer pH 1.2 solution, and tamoxifen in aqueous acetate buffer solution, pH 4.9. First, solubilities of the different compounds in corresponding solvents are measured at temperature 20±1° C. Fluorescein solubility in deionized water is 10±0.5 μg/ml, ibuprofen in HCl 1.2 solution is 22.5±0.5 μg/ml, and for tamoxifen in acetate pH 4.9 is 23.6±0.5 μg/ml.

For dissolution rate experiments a USP 2 stirring apparatus with stirring speed of 100 rpm is used. Concentration is monitored using UV-VIS spectrometer equipped with dipping probe. As an example glass slides substrates with deposited 9 mm diameter drug films are used. Films weights are in the range of 5-80 μg. First, intrinsic dissolution rate (IDR) of films is studied and compared it to dissolution of compressed powder in form of 1.57 mm diameter pellets. IDR is defined as Equation (4):

$$IDR = \frac{{( d m / d t )}_{max}}{A} \qquad ( 4 )$$

In this case (dm/dt)ₘₐₓ is maximum slope in a dissolution curve evaluated at the start of dissolution process (m—dissolved solute mass). Glass substrates with deposited films are attached to a stirring rod having same diameter as compressed pellets rod (20 mm), assuring that hydrodynamic boundary layer thickness is same for compressed powder and deposited film. Solution volume remains constant in all experiments, about 10 ml, and temperature is 20±1° C. In all cases intrinsic dissolution of films is comparable to one of compressed pellets (3×10⁻⁵±5×10⁻⁶ for fluorescein, 1×10⁻³±3×10⁻⁴ for ibuprofen, 6×10⁻⁴±1×10⁻⁴ for tamoxifen, all values in (μg sec⁻¹ mm⁻²))). Since IDR depends on crystal structure and degree of crystallinity of a compound, it indicates that there are no changes in films crystallinity or structure, as was also observed in XRD studies.

FIGS. 10(a)-10(c) show the dissolution behavior of deposited films versus original loose powders. It can be seen that initial dissolution rate in films is very rapid and constant up to ˜80% of the film is dissolved. Further dissolution rate is reduced mainly due to reduction in film surface area. Initial dissolution rates in film versus loose powder are enhanced about ten times for fluorescein (FIG. 10(a)), about 30 times for ibuprofen (FIG. 10(b)) and about 10 times for tamoxifen (FIG. 10(c)). Initial enhancement in dissolution rate is attributed mainly to enhancement of surface area of a film, since IDR or solubility are not changing. The order of enhancement is in good agreement with order of enhancement of powders surface area. Importantly, this example is merely representative of the dissolution improvement that can be achieved when forming pharmaceutical compositions of deposited films in accordance with the present teachings. For instance, if film is dissolving from a soluble polymer substrate, the rate can be further doubled, because dissolution will occur from both sides of the film. Additionally, films dissolution is accurately predictable until almost complete dissolution, whereas in the case of powder, it is more complicated to predict dissolution rate due changes in particles shape and agglomeration, as can be seen in dissolution of ibuprofen powder in FIG. 10(b).

Biological efficacy is also further enhanced from pharmaceutical substances printed from vapor phase, such as tamoxifen and BAY 11-7082. To test drug effectiveness in a deposited film form, cancer cell lines in culture are exposed to tamoxifen films and BAY films deposited on glass slides. See FIG. 11 showing drug application in a film form. The ovarian carcinoma cell line, OVCAR3, and the breast carcinoma cell line, MCF7, are utilized to study growth inhibition in the presence of tamoxifen and BAY-27. Growth inhibition curves are also generated using the following controls: i) Clean glass slides with no deposited drug film as a sham control; ii) 5 μM tamoxifen or 500 nM BAY dissolved in dimethyl sulfoxide (DMSO; conventional drug dose); and iii) tamoxifen or BAY powders dissolved directly in sterile supplemented growth medium. In all cases, the amount of the introduced drug is calculated so the nominal concentration treatment is 5 μM (1.8 μg/ml) for tamoxifen (4.5 μg per film) and 500 nM (0.1 μg/ml) for BAY 11-7082 (0.25 μg per film).

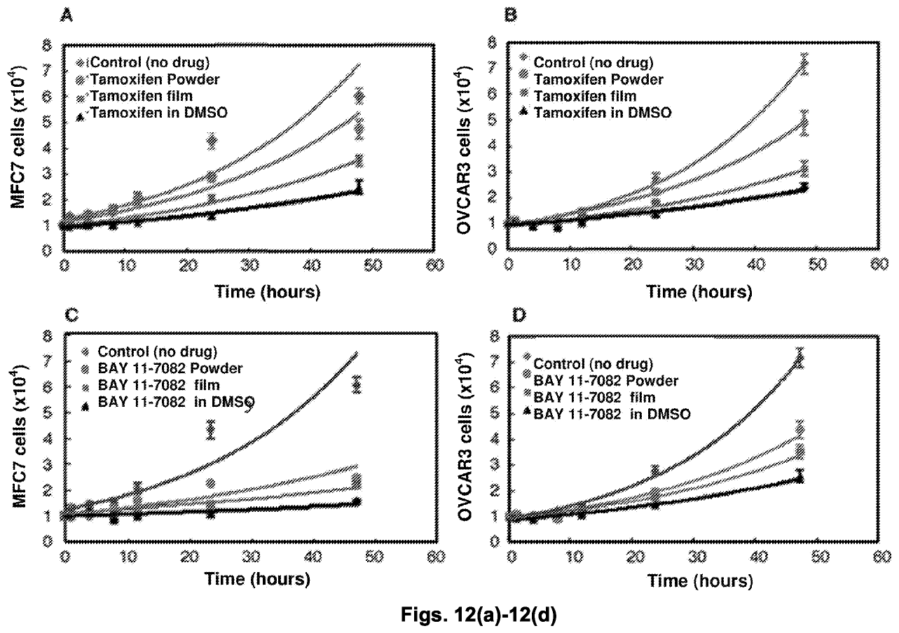

FIGS. 12(a)-12(d) demonstrate cancer cell count curves treated with the different drug forms to demonstrate enhancement in biological efficacy of deposited films prepared in accordance with certain aspects of the present disclosure as compared to a conventional formulation. FIG. 12(a) shows an MCF7 cell treatment curve with tamoxifen (solid line—eye guide). FIG. 12(b) shows an OVCAR3 cell treatment curve with tamoxifen (solid line—eye guide). FIG. 12(c) shows an MCF7 cell treatment curve with BAY 11-7082 (solid line—eye guide). FIG. 12(d) shows OVCAR3 cell treatment curve with BAY 11-7082 (solid line—eye guide).

In both cases, cells treated with film form drug showed almost similar viability to that of drug dissolved in DMSO. Tamoxifen in a film form showed significantly better effectiveness than the powdered drug dissolved in growth medium. MCF7 cancer cells viability after 48 hours was 58% for film form and 79% for powder form (FIG. 12(a)), and OVCAR3 cancer cells viability after 48 hours was 44% for film form and 68% for powder form (FIG. 12(b)). BAY in a film form shows similar effectiveness as powdered drug dissolved in growth medium (FIGS. 12(c)-12(d)).

The reason for difference between powder form and film in tamoxifen is believed to be due to a higher dissolution rate of film as compared to powder form. Because actual concentration of dissolved powder is lower than 5 μM, growth cell inhibition rate is lower. Differences in behavior between tamoxifen and BAY are mainly due to differences in compounds solubility and dissolution rates—tamoxifen solubility at pH 7.4 is less than 5 μg/ml, while solubility of BAY is 29.25±0.05 μg/ml.

In various aspects, the disclosure contemplates high surface area films of small molecular organic compounds, such as medicinal substances, with precise weight and high purity that are fabricated using an organic vapor jet printing deposition technique and apparatus. Further, certain organic compounds, like BAY 11-7082 drug, can be dissolved directly by jetting into a solution and the drug dissolves, having similar efficacy to the same drug dissolved in DMSO. Likewise, direct jetting of fluorescein into phosphate buffer saline solution demonstrated rapid and accurate dissolution of small molecular pharmaceutical substances. These results indicate that organic vapor jet printing deposition techniques can be used to generate pharmaceutical films and particle morphologies with enhanced solubility properties.

All possible combinations discussed and enumerated above and herein as optional features of the inventive materials and inventive methods of the present disclosure are specifically disclosed as embodiments. In various aspects, the present disclosure contemplates a solid film comprising greater than or equal to about 99 mass % of a deposited low molecular weight organic active ingredient compound. The low molecular weight organic active ingredient compound has a molecular weight of less than or equal to about 1,000 g/mol. Further, the low molecular weight organic active ingredient compound is a pharmaceutical active or a new chemical entity. Also specifically disclosed are combinations including this solid film optionally with any one or any combination of more than one of the enumerated features (1)-(17).

The solid film of the first embodiment optionally has any one or any combination of more than one of the following features: (1) a specific surface area of the solid film is greater than or equal to about 0.001 m²/g to less than or equal to about 1,000 m²/g; (2) the deposited low molecular weight organic active ingredient compound in the solid film is amorphous; (3) the amorphous solid film further defines particles having an average particle size of greater than or equal to about 2 nm to less than or equal to about 200 nm; (4) the deposited low molecular weight organic active ingredient compound in the amorphous solid film is stable for greater than or equal to about 1 month; (5) the deposited low molecular weight organic active ingredient compound in the solid film is crystalline or polycrystalline; (6) an average crystal size is greater than or equal to about 2 nm to less than or equal to about 200 nm; (7) the deposited low molecular weight organic active ingredient compound is selected from the group consisting of: anti-proliferative agents; anti-rejection drugs; anti-thrombotic agents; anti-coagulants; antioxidants; free radical scavengers; nucleic acids; saccharides; sugars; nutrients; hormones; cytotoxin; hormonal agonists; hormonal antagonists; inhibitors of hormone biosynthesis and processing; antigestagens; antiandrogens; anti-inflammatory agents; non-steroidal anti-inflammatory agents (NSAIDs); antimicrobial agents; antiviral agents; antifungal agents; antibiotics; chemotherapy agents; antineoplastic/anti-miotic agents; anesthetic, analgesic or pain-killing agents; antipyretic agents, prostaglandin inhibitors; platelet inhibitors; DNA de-methylating agents; cholesterol-lowering agents; vasodilating agents; endogenous vasoactive interference agents; angiogenic substances; cardiac failure active ingredients; targeting toxin agents; and combinations thereof; (8) the deposited low molecular weight organic active ingredient compound is selected from the group consisting of: caffeine, (E)-3-(4-Methylphenylsulfonyl)-2-propenenitrile, fluorescein, paracetamol, ibuprofen, tamoxifen, and combinations thereof; (9) the deposited low molecular weight organic compound has a molecular weight of greater than or equal to about 100 g/mol to less than or equal to about 900 g/mol; (10) an average thickness of the film is less than or equal to about 300 nm and an average surface roughness (Rₐ) is less than or equal to about 100 nm; (11) an average thickness of the film is greater than or equal to about 300 nm and the film defines a nanostructured surface comprising a plurality of nanostructures having a major dimension of greater than or equal to about 5 nm to less than or equal to about 10 μm; (12) the film defines a nanostructured surface comprising a plurality of nanostructures having a shape selected from the group consisting of: needles, tubes, rods, platelets, round particles, droplets, fronds, tree-like structures, fractals, the plurality of nanostructures has a shape selected from the group consisting of: droplet, hemispherical, puddle, interconnected puddle, island, interconnected island, and combinations thereof; (13) comprises one of the following:
a. the deposited low molecular weight organic compound comprises caffeine and the plurality of nanostructures has a needle shape or a tube shape, wherein an average diameter of the plurality of nanostructures is greater than or equal to about 5 nm to less than or equal to about 10 μm and an average length of greater than or equal to about 5 nm to less than or equal to about 100 μm;
b. the deposited low molecular weight organic compound comprises (E)-3-(4-Methylphenylsulfonyl)-2-propenenitrile and the plurality of nanostructures has a platelet shape, wherein an average height of the plurality of nanostructures is greater than or equal to about 10 nm to less than or equal to about 10 μm, an average width of the plurality of nanostructures is greater than or equal to about 5 nm to less than or equal to about 10 μm, and an average length of greater than or equal to about 5 nm to less than or equal to about 100 μm;
c. the deposited low molecular weight organic compound comprises fluorescein and the plurality of nanostructures has a round shape, wherein an average radius of the plurality of nanostructures is greater than or equal to about 5 nm to less than or equal to about 10 μm; or
d. the deposited low molecular weight organic compound comprises paracetamol and the plurality of nanostructures has a shape selected from the group consisting of: droplet, hemispherical, puddle, interconnected puddle, island, interconnected island, and combinations thereof, wherein an average major dimension of the plurality of nanostructures is greater than or equal to about 5 nm to less than or equal to about 20 μm;
   (14) the deposited low molecular weight organic compound has an enhanced rate of dissolution as compared to a comparative powder or pellet form of the low molecular weight organic active ingredient, where a dissolution rate of the deposited low molecular weight organic active ingredient compound in the solid film in an aqueous solution is at least ten times greater than a comparative dissolution rate of the comparative powder or pellet form of the low molecular weight organic active ingredient; (15) the deposited low molecular weight organic compound has an enhanced bioavailability as compared to a comparative powder or pellet form of the low molecular weight organic active ingredient, wherein a bioavailability of the deposited low molecular weight organic active ingredient compound in the solid film is at least about 10% greater than a comparative bioavailability of the comparative powder or pellet form of the low molecular weight organic active ingredient; (16) the solid film is substantially free of any binders or impurities; and/or (17) the solid film comprises greater than or equal to about 99.5 mass % of the deposited low molecular weight organic active ingredient compound.

In other aspects, the present disclosure contemplates a second embodiment that is an article comprising a surface of a solid substrate having one or more discrete regions patterned with a deposited low molecular weight organic compound having a molecular weight of less than or equal to about 1,000 g/mol. The deposited low molecular weight organic compound is present at greater than or equal to about 99 mass % in the one or more discrete regions. Also specifically disclosed are combinations including this article optionally with any one or any combination of more than one of the enumerated features (18)-(34) or any of the previous enumerated features (1)-(17).

The article of the second embodiment optionally has any one or any combination of more than one of the following features: (18) a specific surface area of the deposited low molecular weight organic compound in the one or more discrete regions is greater than or equal to about 0.001 m²/g to less than or equal to about 1,000 m²/g; (19) the deposited low molecular weight organic compound is amorphous; (20) the amorphous low molecular weight organic compound further defines particles having an average particle size of greater than or equal to about 2 nm to less than or equal to about 200 nm; (21) the deposited low molecular weight organic compound is stable for greater than or equal to about 1 month; (22) the deposited low molecular weight organic compound is crystalline or polycrystalline; (23) an average crystal size is greater than or equal to about 2 nm to less than or equal to about 200 nm; (24) the deposited low molecular weight organic compound is selected from the group consisting of: anti-proliferative agents; anti-rejection drugs; anti-thrombotic agents; anti-coagulants; antioxidants; free radical scavengers; nucleic acids; saccharides; sugars; nutrients; hormones; cytotoxin; hormonal agonists; hormonal antagonists; inhibitors of hormone biosynthesis and processing; antigestagens; antiandrogens; anti-inflammatory agents; non-steroidal anti-inflammatory agents (NSAIDs); antimicrobial agents; antiviral agents; antifungal agents; antibiotics; chemotherapy agents; antineoplastic/anti-miotic agents; anesthetic, analgesic or pain-killing agents; antipyretic agents, prostaglandin inhibitors; platelet inhibitors; DNA de-methylating agents; cholesterol-lowering agents; vasodilating agents; endogenous vasoactive interference agents; angiogenic substances; cardiac failure active ingredients; targeting toxin agents; and combinations thereof; (25) the deposited low molecular weight organic compound is selected from the group consisting of: caffeine, (E)-3-(4-Methylphenylsulfonyl)-2-propenenitrile, fluorescein, paracetamol, ibuprofen, tamoxifen, and combinations thereof; (26) the deposited low molecular weight organic compound has a molecular weight of greater than or equal to about 100 g/mol to less than or equal to about 900 g/mol; (27) an average thickness of the deposited low molecular weight organic compound is less than or equal to about 300 nm and an average surface roughness (Rₐ) is less than or equal to about 100 nm; (28) an average thickness of the deposited low molecular weight organic compound is greater than or equal to about 300 nm and the film defines a nanostructured surface comprising a plurality of nanostructures having a major dimension of greater than or equal to about 5 nm to less than or equal to about 10 μm; (29) the deposited low molecular weight organic compound defines a nanostructured surface comprising a plurality of nanostructures having a shape selected from the group consisting of: needles, tubes, rods, platelets, round particles, droplets, fronds, tree-like structures, fractals, hemispheres, puddles, interconnected puddles, islands, interconnected islands, and combinations thereof; (30) comprises one of the following:
a. the deposited low molecular weight organic compound comprises caffeine and the plurality of nanostructures has a needle shape or a tube shape, wherein an average diameter of the plurality of nanostructures is greater than or equal to about 5 nm to less than or equal to about 10 μm and an average length of greater than or equal to about 5 nm to less than or equal to about 100 μm;
b. the deposited low molecular weight organic compound comprises (E)-3-(4-Methylphenylsulfonyl)-2-propenenitrile and the plurality of nanostructures has a platelet shape, wherein an average height of the plurality of nanostructures is greater than or equal to about 10 nm to less than or equal to about 10 μm, an average width of the plurality of nanostructures is greater than or equal to about 5 nm to less than or equal to about 10 μm, and an average length of greater than or equal to about 5 nm to less than or equal to about 100 μm;
c. the deposited low molecular weight organic compound comprises fluorescein and the plurality of nanostructures has a round shape, wherein an average radius of the plurality of nanostructures is greater than or equal to about 5 nm to less than or equal to about 10 μm; or
d. the deposited low molecular weight organic compound comprises paracetamol and the plurality of nanostructures has a shape selected from the group consisting of: droplet, hemispherical, puddle, interconnected puddle, island, interconnected island, and combinations thereof, wherein an average major dimension of the plurality of nanostructures is greater than or equal to about 5 nm to less than or equal to about 20 μm;
   (31) the deposited low molecular weight organic compound has an enhanced rate of dissolution as compared to a comparative powder or pellet form of the low molecular weight organic active ingredient, where a dissolution rate of the deposited low molecular weight organic active ingredient compound in the solid film in an aqueous solution is at least ten times greater than a comparative dissolution rate of the comparative powder or pellet form of the low molecular weight organic active ingredient; (32) the deposited low molecular weight organic compound has an enhanced bioavailability as compared to a comparative powder or pellet form of the low molecular weight organic active ingredient, wherein a bioavailability of the deposited low molecular weight organic active ingredient compound in the solid film is at least about 10% greater than a comparative bioavailability of the comparative powder or pellet form of the low molecular weight organic active ingredient; (33) the deposited low molecular weight organic compound is substantially free of any binders or impurities; and/or (34) the one or more discrete regions comprise greater than or equal to about 99.5 mass % of the deposited low molecular weight organic active ingredient compound.

In other aspects, the present disclosure contemplates a third embodiment that is an article comprising a pharmaceutically acceptable substrate defining a surface and a deposited solid low molecular weight pharmaceutical active ingredient having a molecular weight of less than or equal to about 1,000 g/mol. The deposited solid low molecular weight pharmaceutical active ingredient is present at greater than or equal to about 99 mass % in one or more discrete regions on the surface of the pharmaceutically acceptable substrate.

Also specifically disclosed are combinations including this article optionally with any one or any combination of more than one of the enumerated features (35)-(55) or any of the previous enumerated features (1)-(34).

The article of the third embodiment optionally has any one or any combination of more than one of the following features: (35) the one or more discrete regions of the surface are continuous and the deposited solid low molecular weight pharmaceutical active ingredient forms a solid film on the surface of the pharmaceutically acceptable substrate; (36) the pharmaceutically acceptable substrate is biodegradable; (37) the pharmaceutically acceptable substrate comprises a pharmaceutically acceptable material selected from the group consisting of: glass, metals, siloxanes, polymers, hydrogels, organogels, organic materials, natural fibers, synthetic fibers, ceramic, biological tissue, and combinations thereof; (38) the pharmaceutically acceptable substrate is selected from the group consisting of: a microneedle, medical equipment, an implant, a film, a gel, a patch, a dressing, a fabric, a bandage, a sponge, a stent, a contact lens, a subretinal implant prosthesis, dentures, braces, a wearable device, a bracelet, and combinations thereof; (39) a specific surface area of deposited solid low molecular weight pharmaceutical active ingredient in the one or more discrete regions is greater than or equal to about 0.001 m²/g to less than or equal to about 1,000 m²/g; (40) the deposited solid low molecular weight pharmaceutical active ingredient is amorphous; (41) the amorphous deposited solid low molecular weight pharmaceutical active ingredient further defines particles having an average particle size of greater than or equal to about 2 nm to less than or equal to about 200 nm; (42) the amorphous deposited solid low molecular weight pharmaceutical active ingredient is stable for greater than or equal to about 1 month; (43) the deposited solid low molecular weight pharmaceutical active ingredient is crystalline or polycrystalline; (44) an average crystal size is greater than or equal to about 2 nm to less than or equal to about 200 nm; (45) the deposited solid low molecular weight pharmaceutical active ingredient is selected from the group consisting of: anti-proliferative agents; anti-rejection drugs; anti-thrombotic agents; anti-coagulants; antioxidants; free radical scavengers; nucleic acids; saccharides; sugars; nutrients; hormones; cytotoxin; hormonal agonists; hormonal antagonists; inhibitors of hormone biosynthesis and processing; antigestagens; antiandrogens; anti-inflammatory agents; non-steroidal anti-inflammatory agents (NSAIDs); antimicrobial agents; antiviral agents; antifungal agents; antibiotics; chemotherapy agents; antineoplastic/anti-miotic agents; anesthetic, analgesic or pain-killing agents; antipyretic agents, prostaglandin inhibitors; platelet inhibitors; DNA de-methylating agents; cholesterol-lowering agents; vasodilating agents; endogenous vasoactive interference agents; angiogenic substances; cardiac failure active ingredients; targeting toxin agents; and combinations thereof; (46) the deposited solid low molecular weight pharmaceutical active ingredient is selected from the group consisting of: caffeine, (E)-3-(4-Methylphenylsulfonyl)-2-propenenitrile, fluorescein, paracetamol, ibuprofen, tamoxifen, and combinations thereof; (47) the molecular weight of the deposited solid low molecular weight pharmaceutical active ingredient is greater than or equal to about 100 g/mol to less than or equal to about 900 g/mol; (48) an average thickness of the deposited solid low molecular weight pharmaceutical active ingredient in the one or more discrete regions is less than or equal to about 300 nm and an average surface roughness (Rₐ) is less than or equal to about 100 nm; (49) an average thickness of the deposited solid low molecular weight pharmaceutical active ingredient in the one or more discrete regions is greater than or equal to about 300 nm and the deposited solid low molecular weight pharmaceutical active ingredient defines a nanostructured surface comprising a plurality of nanostructures having a major dimension of greater than or equal to about 5 nm to less than or equal to about 10 μm; (50) the deposited solid low molecular weight pharmaceutical active ingredient defines a nanostructured surface having a plurality of nanostructures with a shape selected from the group consisting of: needles, tubes, rods, platelets, round particles, droplets, fronds, tree-like structures, fractals, hemispheres, puddles, interconnected puddles, islands, interconnected islands, and combinations thereof; (51) comprises one of the following:
a. the deposited solid low molecular weight pharmaceutical active ingredient comprises caffeine and the plurality of nanostructures has a needle shape or a tube shape, wherein an average diameter of the plurality of nanostructures is greater than or equal to about 5 nm to less than or equal to about 10 μm and an average length of greater than or equal to about 5 nm to less than or equal to about 100 μm;
b. the deposited solid low molecular weight pharmaceutical active ingredient comprises (E)-3-(4-Methylphenylsulfonyl)-2-propenenitrile and the plurality of nanostructures has a platelet shape, wherein an average height of the plurality of nanostructures is greater than or equal to about 10 nm to less than or equal to about 10 μm, an average width of the plurality of nanostructures is greater than or equal to about 5 nm to less than or equal to about 10 μm, and an average length of greater than or equal to about 5 nm to less than or equal to about 100 μm;
c. the deposited solid low molecular weight pharmaceutical active ingredient comprises fluorescein and the plurality of nanostructures has a round shape, wherein an average radius of the plurality of nanostructures is greater than or equal to about 5 nm to less than or equal to about 10 μm; or
d. the deposited solid low molecular weight pharmaceutical active ingredient comprises paracetamol and the plurality of nanostructures has a shape selected from the group consisting of: droplet, hemispherical, puddle, interconnected puddle, island, interconnected island, and combinations thereof, wherein an average major dimension of the plurality of nanostructures is greater than or equal to about 5 nm to less than or equal to about 20 μm;
   (52) the deposited solid low molecular weight pharmaceutical active ingredient has an enhanced rate of dissolution as compared to a comparative powder or pellet form of the low molecular weight pharmaceutical active ingredient, where a dissolution rate of the deposited solid low molecular weight pharmaceutical active ingredient in an aqueous solution is at least ten times greater than a comparative dissolution rate of the comparative powder or pellet form of the low molecular weight pharmaceutical active ingredient; (53) the deposited solid low molecular weight pharmaceutical active ingredient has an enhanced bioavailability as compared to a comparative powder or pellet form of the low molecular weight pharmaceutical active ingredient, wherein a bioavailability of the deposited low molecular weight organic active ingredient compound in the solid film is at least about 10% greater than a comparative bioavailability of the comparative powder or pellet form of the low molecular weight pharmaceutical active ingredient; (54) the deposited solid low molecular weight pharmaceutical active ingredient is substantially free of any binders or impurities; and/or (55) the one or more discrete regions comprise greater than or equal to about 99.5 mass % of the deposited solid low molecular weight pharmaceutical active ingredient.

In other aspects, the present disclosure contemplates a fourth embodiment that is an article comprising a solid deposited film comprising a pharmaceutical composition comprising at least one low molecular weight organic compound having a molecular weight of less than or equal to about 1,000 g/mol. Also specifically disclosed are combinations including this article optionally with any one or any combination of more than one of the enumerated features (56)-(68) or any of the previous enumerated features (1)-(55).

The article of the fourth embodiment optionally has any one or any combination of more than one of the following features: (56) the pharmaceutical composition further comprises at least one additional deposited compound distinct from the low molecular weight organic compound; (57) the pharmaceutical composition comprises at least two low molecular weight organic compounds; (58) the pharmaceutical composition has at least one low molecular weight organic compound present at greater than or equal to about 99 mass % in the solid deposited film; (59) the article is a multilayered stack and the solid deposited film comprising the pharmaceutical composition is a first layer and the multilayered stack comprises a second layer having a distinct chemical composition; (60) the second layer comprises a second distinct pharmaceutical composition from pharmaceutical composition in the first layer; (61) the second layer comprises a material that minimizes dissolution rate of the pharmaceutical composition in the first layer; (62) the second layer comprises a material having a solubility controlled by the presence of a trigger selected from the group consisting of: light, radiation, magnetism, radio waves, pH of a surrounding medium, and combinations thereof; (63) a specific surface area of the solid deposited film is greater than or equal to about 0.001 m²/g to less than or equal to about 1,000 m²/g; (64) the solid deposited film is stable for greater than or equal to about 1 month; (65) the low molecular weight organic compound is a pharmaceutical active ingredient or a new chemical entity selected from the group consisting of: anti-proliferative agents; anti-rejection drugs; anti-thrombotic agents; anti-coagulants; antioxidants; free radical scavengers; nucleic acids; saccharides; sugars; nutrients; hormones; cytotoxin; hormonal agonists; hormonal antagonists; inhibitors of hormone biosynthesis and processing; antigestagens; antiandrogens; anti-inflammatory agents; non-steroidal anti-inflammatory agents (NSAIDs); antimicrobial agents; antiviral agents; antifungal agents; antibiotics; chemotherapy agents; antineoplastic/anti-miotic agents; anesthetic, analgesic or pain-killing agents; antipyretic agents, prostaglandin inhibitors; platelet inhibitors; DNA de-methylating agents; cholesterol-lowering agents; vasodilating agents; endogenous vasoactive interference agents; angiogenic substances; cardiac failure active ingredients; targeting toxin agents; and combinations thereof; (66) the low molecular weight organic compound is selected from the group consisting of: caffeine, (E)-3-(4-Methylphenylsulfonyl) propenenitrile, fluorescein, paracetamol, ibuprofen, tamoxifen, and combinations thereof; (67) the low molecular weight organic compound in the pharmaceutical composition has an enhanced solubility as compared to a comparative powder or pellet form of low molecular weight organic compound, wherein a dissolution rate of the low molecular weight organic compound in an aqueous solution is at least ten times greater than a dissolution rate of the comparative powder or pellet form of the low molecular weight organic compound; (68) the deposited low molecular weight organic compound in the pharmaceutical composition has an enhanced bioavailability as compared to a comparative powder or pellet form of the low molecular weight organic active ingredient, wherein a bioavailability of the deposited low molecular weight organic active ingredient compound in the pharmaceutical composition is at least about 10% greater than a comparative bioavailability of the comparative powder or pellet form of the low molecular weight organic active ingredient.

In other aspects, the present disclosure contemplates a fifth embodiment of a method for solvent-free vapor deposition. The method comprises depositing a low molecular weight organic compound having a molecular weight of less than or equal to about 1,000 g/mol on one or more discrete regions of a substrate in a process that is substantially free of solvents. The process is selected from the group consisting of: vacuum thermal evaporation (VTE), organic vapor jet printing (OVJP), organic vapor phase deposition (OVPD), organic molecular beam deposition (OMBD), molecular jet printing (MoJet), and organic vapor jet printing (OVJP), and organic vapor phase deposition (OVPD). A deposited low molecular weight organic compound is present at greater than or equal to about 99 mass % in the one or more discrete regions.

Also specifically disclosed are combinations including this method optionally with any one or any combination of more than one of the enumerated steps or features (69)-(91) or any of the previous enumerated features (1)-(68) in the context of the first through fourth embodiments. The method for solvent-free vapor deposition optionally has any one or any combination of more than one of the following steps or features: (69) further comprising entraining the low molecular weight organic compound in an inert gas stream or vacuum that is substantially free of any solvents prior to the depositing; (70) wherein prior to the entraining, the low molecular weight organic compound is in a form selected from the group consisting of: a powder, a pressed pellet, a porous material, and a liquid; (71) wherein prior to the entraining, the low molecular weight organic compound is dispersed in a porous material; (72) wherein prior to the entraining, the low molecular weight organic compound is dispersed in a liquid bubbler through which the inert gas stream passes; (73) the entraining of the low molecular weight organic compound in the inert gas stream or vacuum is conducted by heating a source of a solid low molecular weight organic compound to sublimate or evaporate the low molecular weight organic compound; (74) the low molecular weight organic compound is deposited onto the one or more discrete regions at a loading density of greater than or equal to about 1×10⁻⁴ g/cm² to less than or equal to about 1 g/cm²; (75) a parameter is adjusted to affect a morphology, a degree of crystallinity, or both the morphology and the degree of crystallinity of the deposited low molecular weight organic compound, wherein the parameter is selected from the group consisting of: system pressure, a flow rate of the inert gas stream, a composition of the inert gas, a temperature of a source of the low molecular weight organic compound, a composition of the substrate, a surface texture of the substrate, a temperature of the substrate, and combinations thereof; (76) a specific surface area of the deposited low molecular weight organic compound is greater than or equal to about 0.001 m²/g to less than or equal to about 1,000 m²/g; (77) the deposited low molecular weight organic compound is amorphous; (78) the deposited low molecular weight organic compound further defines particles having an average particle size of greater than or equal to about 2 nm to less than or equal to about 200 nm; (79) the deposited low molecular weight organic compound is crystalline or polycrystalline; (80) an average crystal size is greater than or equal to about 2 nm to less than or equal to about 200 nm; (81) the deposited low molecular weight organic compound is a pharmaceutical active ingredient or a new chemical entity selected from the group consisting of: anti-proliferative agents; anti-rejection drugs; anti-thrombotic agents; anti-coagulants; antioxidants; free radical scavengers; nucleic acids; saccharides; sugars; nutrients; hormones; cytotoxin; hormonal agonists; hormonal antagonists; inhibitors of hormone biosynthesis and processing; antigestagens; antiandrogens; anti-inflammatory agents; non-steroidal anti-inflammatory agents (NSAIDs); antimicrobial agents; antiviral agents; antifungal agents; antibiotics; chemotherapy agents; antineoplastic/anti-miotic agents; anesthetic, analgesic or pain-killing agents; antipyretic agents, prostaglandin inhibitors; platelet inhibitors; DNA de-methylating agents; cholesterol-lowering agents; vasodilating agents; endogenous vasoactive interference agents; angiogenic substances; cardiac failure active ingredients; targeting toxin agents; and combinations thereof; (82) the low molecular weight organic compound is selected from the group consisting of: caffeine, (E)-3-(4-Methylphenylsulfonyl)-2-propenenitrile, fluorescein, paracetamol, ibuprofen, tamoxifen, and combinations thereof; (83) the molecular weight of the deposited low molecular weight organic active ingredient compound is greater than or equal to about 100 g/mol to less than or equal to about 900 g/mol; (84) an average thickness of the deposited low molecular weight organic compound in the one or more discrete regions is less than or equal to about 300 nm and an average surface roughness (Rₐ) is less than or equal to about 100 nm; (85) an average thickness of the deposited low molecular weight organic compound in the one or more discrete regions is greater than or equal to about 300 nm and the deposited low molecular weight organic compound defines a nanostructured surface having a plurality of nanostructures having a major dimension of greater than or equal to about 5 nm to less than or equal to about 10 μm; (86) the plurality of nanostructures has a shape selected from the group consisting of: needles, tubes, rods, platelets, round particles, droplets, fronds, tree-like structures, fractals, hemispheres, puddles, interconnected puddles, islands, interconnected islands, and combinations thereof; (87) where a purity level of the deposited low molecular weight organic compound in the one or more discrete regions is greater than or equal to about 99.5 mass %; (88) the low molecular weight organic compound is a pharmaceutical active ingredient or a new chemical entity; (89) the one or more discrete regions are continuous and the deposited low molecular weight organic compound forms a solid film on the surface of the substrate; (90) the deposited low molecular weight organic compound has an enhanced rate of dissolution as compared to a comparative powder or pellet form of the deposited low molecular weight organic compound, wherein a dissolution rate of the deposited low molecular weight organic compound in an aqueous solution is at least ten times greater than a dissolution rate of the comparative powder or pellet form of the deposited low molecular weight organic compound; (91) the deposited low molecular weight organic compound has an enhanced bioavailability as compared to a comparative powder or pellet form of the low molecular weight organic active ingredient, wherein a bioavailability of the deposited low molecular weight organic active ingredient compound is at least about 10% greater than a comparative bioavailability of the comparative powder or pellet form of the low molecular weight organic active ingredient.

In other aspects, the present disclosure contemplates a sixth embodiment of a method for an organic vapor jet printing deposition. The method comprises entraining a low molecular weight organic compound in an inert gas stream by heating a source of a solid low molecular weight organic compound to sublimate the low molecular weight organic compound. The inert gas stream is passed over, by, or through the source. The low molecular weight organic compound is entrained in the inert gas stream through a nozzle towards a cooled target. The low molecular weight organic compound is condensed as it contacts the cooled target.

Also specifically disclosed are combinations including this method optionally with any one or any combination of more than one of the enumerated steps or features (92)-(108) or any of the previous enumerated features (1)-(91). The method for organic vapor jet printing deposition optionally has any one or any combination of more than one of the following steps or features: (92) the cooled target is a surface of a substrate and the condensed low molecular weight organic compound is deposited on one or more discrete regions of the surface; (93) the condensed low molecular weight organic compound is deposited onto the one or more discrete regions of the surface at a loading density of greater than or equal to about 1×10⁻⁴ g/cm² to less than or equal to about 1 g/cm²; (94) a specific surface area of the condensed low molecular weight organic compound in the one or more discrete regions is greater than or equal to about 0.001 m²/g to less than or equal to about 1000 m²/g; (95) an average thickness of the condensed low molecular weight organic compound in the one or more discrete regions is less than or equal to about 300 nm and an average surface roughness (Rₐ) is less than or equal to about 100 nm; (96) an average thickness of the condensed low molecular weight organic compound in the one or more discrete regions is greater than or equal to about 300 nm and the condensed low molecular weight organic compound defines a nanostructured surface having a plurality of nanostructures having a major dimension of greater than or equal to about 5 nm to less than or equal to about 10 μm; (97) the plurality of nanostructures has a shape selected from the group consisting of: needles, tubes, rods, platelets, round particles, droplets, fronds, tree-like structures, fractals, hemispheres, puddles, interconnected puddles, islands, interconnected islands, and combinations thereof; (98) the one or more discrete regions of the surface are continuous and the condensed low molecular weight organic compound forms a solid film on the surface of the substrate; (99) a purity level of the condensed low molecular weight organic compound is greater than or equal to about 99.5 mass %; (100) the cooled target is a liquid comprising one or more solvents; (101) the entraining and directing are conducted at atmospheric pressure conditions; (102) wherein the entraining and directing are conducted at reduced pressure conditions of greater than or equal to about 0.1 Torr to less than or equal to about 500 Torr; (103) a parameter is adjusted to affect a morphology, a degree of crystallinity, or both the morphology and the degree of crystallinity of the condensed low molecular weight organic compound, wherein the parameter is selected from the group consisting of: system pressure, flow rate of the inert gas stream, inert gas composition, a temperature of the source, a composition of a target substrate, a surface texture of the target substrate, a temperature of the target substrate, and combinations thereof; (104) wherein the condensed low molecular weight organic compound is amorphous; (105) wherein the condensed low molecular weight organic compound is crystalline or polycrystalline; (106) wherein the low molecular weight organic compound is a pharmaceutical active or a new chemical entity selected from the group consisting of: anti-proliferative agents; anti-rejection drugs; anti-thrombotic agents; anti-coagulants; antioxidants; free radical scavengers; nucleic acids; saccharides; sugars; nutrients; hormones; cytotoxin; hormonal agonists; hormonal antagonists; inhibitors of hormone biosynthesis and processing; antigestagens; antiandrogens; anti-inflammatory agents; non-steroidal anti-inflammatory agents (NSAIDs); antimicrobial agents; antiviral agents; antifungal agents; antibiotics; chemotherapy agents; antineoplastic/anti-miotic agents; anesthetic, analgesic or pain-killing agents; antipyretic agents, prostaglandin inhibitors; platelet inhibitors; DNA de-methylating agents; cholesterol-lowering agents; vasodilating agents; endogenous vasoactive interference agents; angiogenic substances; cardiac failure active ingredients; targeting toxin agents; and combinations thereof; (107) wherein the low molecular weight organic compound is selected from the group consisting of: caffeine, (E)-3-(4-Methylphenylsulfonyl)-2-propenenitrile, fluorescein, paracetamol, ibuprofen, tamoxifen, and combinations thereof; and/or (108) wherein the low molecular weight organic compound is a pharmaceutical active or a new chemical entity and has a molecular weight of greater than or equal to about 100 g/mol to less than or equal to about 900 g/mol.

In other aspects, the present disclosure contemplates a seventh embodiment of a method for rapid dissolution of low molecular weight organic compounds. The method comprises passing a gas stream comprising an inert gas past a heated source of the low molecular weight organic compound. The low molecular weight organic compound is volatilized and entrained in the gas stream. The method also involves depositing the low molecular weight organic compound into a liquid comprising one or more solvents by passing the gas stream through a nozzle towards the liquid, so that the deposited low molecular weight organic compound is dissolved in the liquid.

Also specifically disclosed are combinations including this method optionally with any one or any combination of more than one of the enumerated steps or features (109)-(121) or any of the previous enumerated features (1)-(108). The method for rapid dissolution of low molecular weight organic compounds optionally has any one or any combination of more than one of the following steps or features: (109) the heated source comprises a porous ceramic holder comprising the low molecular weight organic compound that receives heat transferred from a heater; (110) the heated source has a temperature of greater than or equal to about 250° C. and the liquid is at ambient temperature; (111) the nozzle is greater than or equal to about 15 mm to less than or equal to about 25 mm from a surface of the liquid; (112) the inert gas comprises nitrogen; (113) the liquid is an aqueous liquid comprising water; (114) a concentration of the low molecular weight organic compound is greater than or equal to about 1×10⁻¹¹ mol/L to less than or equal to about 20 mol/L; (115) an amount of low molecular weight organic compound deposited is less than or equal to about 100 μg; (116) a volume of the liquid is less than or equal to about 100 ml; (117) the depositing is conducted for greater than or equal to about 1 minute to less than or equal to about 120 minutes; (118) the low molecular weight organic compound is a pharmaceutical active or a new chemical entity selected from the group consisting of: anti-proliferative agents; anti-rejection drugs; anti-thrombotic agents; anti-coagulants; antioxidants; free radical scavengers; nucleic acids; saccharides; sugars; nutrients; hormones; cytotoxin; hormonal agonists; hormonal antagonists; inhibitors of hormone biosynthesis and processing; antigestagens; antiandrogens; anti-inflammatory agents; non-steroidal anti-inflammatory agents (NSAIDs); antimicrobial agents; antiviral agents; antifungal agents; antibiotics; chemotherapy agents; antineoplastic/anti-miotic agents; anesthetic, analgesic or pain-killing agents; antipyretic agents, prostaglandin inhibitors; platelet inhibitors; DNA de-methylating agents; cholesterol-lowering agents; vasodilating agents; endogenous vasoactive interference agents; angiogenic substances; cardiac failure active ingredients; targeting toxin agents; and combinations thereof; (119) the low molecular weight organic compound is selected from the group consisting of: caffeine, (E)-3-(4-Methylphenylsulfonyl)-2-propenenitrile, fluorescein, paracetamol, ibuprofen, tamoxifen, and combinations thereof; (120) the low molecular weight organic compound is a pharmaceutical active ingredient or a new chemical entity having a molecular weight of greater than or equal to about 100 g/mol to less than or equal to about 1,000 g/mol; and/or (121) the low molecular weight organic compound is a pharmaceutical active ingredient or a new chemical entity having a molecular weight of greater than or equal to about 100 g/mol to less than or equal to about 900 g/mol.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

1.-72. (canceled)

73. A solvent-free vapor deposition method comprising:
depositing a low molecular weight organic compound having a molecular weight of less than or equal to about 1,000 g/mol on one or more discrete regions of a substrate in a process that is substantially free of solvents selected from the group consisting of: vacuum thermal evaporation (VTE), organic vapor jet printing (OVJP), organic molecular beam deposition (OMBD), molecular jet printing (MoJet), and organic vapor phase deposition (OVPD), wherein a deposited low molecular weight organic compound is present at greater than or equal to about 99 mass % in the one or more discrete regions.

74. The solvent-free vapor deposition method of claim 73, further comprising entraining the low molecular weight organic compound in an inert gas stream or vacuum that is substantially free of any solvents prior to the depositing.

75.-77. (canceled)

79. The solvent-free vapor deposition method of claim 74, wherein the low molecular weight organic compound is deposited onto the one or more discrete regions at a loading density of greater than or equal to about 1×10⁻⁴ g/cm² to less than or equal to about 1 g/cm².

80. The solvent-free vapor deposition method of claim 74, wherein a parameter is adjusted to affect a morphology, a degree of crystallinity, or both the morphology and the degree of crystallinity of the deposited low molecular weight organic compound, wherein the parameter is selected from the group consisting of: system pressure, a flow rate of the inert gas stream, a composition of the inert gas, a temperature of a source of the low molecular weight organic compound, a composition of the substrate, a surface texture of the substrate, a temperature of the substrate, and combinations thereof.

81. The solvent-free vapor deposition method of claim 73, wherein a specific surface area of the deposited low molecular weight organic compound is greater than or equal to about 0.001 m²/g to less than or equal to about 1,000 m²/g.

82. The solvent-free vapor deposition method of claim 73, wherein the deposited low molecular weight organic compound is amorphous, crystalline, or polycrystalline.

83. The solvent-free vapor deposition method of claim 82, wherein the deposited low molecular weight organic compound further defines particles having an average particle size of greater than or equal to about 2 nm to less than or equal to about 200 nm or an average crystal size is greater than or equal to about 2 nm to less than or equal to about 200 nm.

84.-88. (canceled)

89. The solvent-free vapor deposition method of claim 73, wherein
(i) an average thickness of the deposited low molecular weight organic compound in the one or more discrete regions is less than or equal to about 300 nm and an average surface roughness (Rₐ) is less than or equal to about 100 nm; or
(ii) an average thickness of the deposited low molecular weight organic compound in the one or more discrete regions is greater than or equal to about 300 nm and the deposited low molecular weight organic compound defines a nanostructured surface having a plurality of nanostructures having a major dimension of greater than or equal to about 5 nm to less than or equal to about 10 μm.

90.-94. (canceled)

95. The solvent-free vapor deposition method of claim 73, wherein
(i) the deposited low molecular weight organic compound has an enhanced rate of dissolution as compared to a comparative powder or pellet form of the deposited low molecular weight organic compound, wherein a dissolution rate of the deposited low molecular weight organic compound in an aqueous solution is at least ten times greater than a dissolution rate of the comparative powder or pellet form of the deposited low molecular weight organic compound; or
(ii) the deposited low molecular weight organic compound has an enhanced bioavailability as compared to a comparative powder or pellet form of the low molecular weight organic compound, wherein a bioavailability of the deposited low molecular weight organic compound is at least about 10% greater than a comparative bioavailability of the comparative powder or pellet form of the low molecular weight organic compound.

96. (canceled)

97. An organic vapor jet printing deposition method comprising:
entraining a low molecular weight organic compound in an inert gas stream by heating a source of a solid low molecular weight organic compound to sublimate the low molecular weight organic compound and passing the inert gas stream over, by, or through the source;
directing the low molecular weight organic compound in the inert gas stream through a nozzle towards a cooled target; and
condensing the low molecular weight organic compound as it contacts the cooled target.

98. The organic vapor jet printing deposition method of claim 97, wherein the cooled target is a surface of a substrate and a condensed low molecular weight organic compound is deposited on one or more discrete regions of the surface, or the cooled target is a liquid comprising one or more solvents.

99. The organic vapor jet printing deposition method of claim 98, wherein:
(i) the condensed low molecular weight organic compound is deposited onto the one or more discrete regions of the surface at a loading density of greater than or equal to about 1×10⁻⁴ g/cm² to less than or equal to about 1 g/cm²;
(ii) a specific surface area of the condensed low molecular weight organic compound in the one or more discrete regions is greater than or equal to about 0.001 m²/g to less than or equal to about 1000 m²/g;
(iii) an average thickness of the condensed low molecular weight organic compound in the one or more discrete regions is less than or equal to about 300 nm and an average surface roughness (Ra) is less than or equal to about 100 nm; or
(iv) an average thickness of the condensed low molecular weight organic compound in the one or more discrete regions is greater than or equal to about 300 nm and the condensed low molecular weight organic compound defines a nanostructured surface having a plurality of nanostructures having a major dimension of greater than or equal to about 5 nm to less than or equal to about 10 μm.

100.-106. (canceled)

107. The organic vapor jet printing deposition method of claim 97, wherein the entraining and directing are conducted at atmospheric pressure conditions or at reduced pressure conditions of greater than or equal to about 0.1 Torr to less than or equal to about 500 Torr.

108. (canceled)

109. The organic vapor jet printing deposition method of claim 97, wherein a parameter is adjusted to affect a morphology, a degree of crystallinity, or both the morphology and the degree of crystallinity of a condensed low molecular weight organic compound, wherein the parameter is selected from the group consisting of: system pressure, flow rate of the inert gas stream, inert gas composition, a temperature of the source, a composition of a target substrate, a surface texture of the target substrate, a temperature of the target substrate, and combinations thereof.

110.-114. (canceled)

115. A method for rapid dissolution of low molecular weight organic compounds, the method comprising:
passing a gas stream comprising an inert gas past a heated source of the low molecular weight organic compound, wherein the low molecular weight organic compound is volatilized and entrained in the gas stream; and
depositing the low molecular weight organic compound into a liquid comprising one or more solvents by passing the gas stream through a nozzle towards the liquid, so that a deposited low molecular weight organic compound is dissolved in the liquid.

116.-127. (canceled)

128. A solid film comprising greater than or equal to about 99 mass % of a deposited low molecular weight organic active ingredient compound having a molecular weight of less than or equal to about 1,000 g/mol, wherein the low molecular weight organic active ingredient compound is a pharmaceutical active or a new chemical entity, wherein the solid film is produced by the solvent-free vapor deposition method of claim 73.

129. An article comprising: a surface of a solid substrate having one or more discrete regions patterned with a deposited low molecular weight organic compound having a molecular weight of less than or equal to about 1,000 g/mol, wherein the deposited low molecular weight organic compound is present at greater than or equal to about 99 mass % in the one or more discrete regions, wherein the article is produced by the solvent-free vapor deposition method of claim 73.

130. A solid film comprising greater than or equal to about 99 mass % of a deposited low molecular weight organic active ingredient compound having a molecular weight of less than or equal to about 1,000 g/mol, wherein the low molecular weight organic active ingredient compound is a pharmaceutical active or a new chemical entity, wherein the solid film is produced by the organic vapor jet printing deposition method of claim 97.

131. An article comprising: a surface of a solid substrate having one or more discrete regions patterned with a deposited low molecular weight organic compound having a molecular weight of less than or equal to about 1,000 g/mol, wherein the deposited low molecular weight organic compound is present at greater than or equal to about 99 mass % in the one or more discrete regions, wherein the article is produced by the organic vapor jet printing deposition method of claim 97.
